(19) United States
(12) Patent Application Publication
(10) Pub. No.: US 2008/0199726 A1
(43) Pub. Date: Aug. 21, 2008

(54) Electroluminescent Device

(75) Inventors: Thomas Schafer, Liestal (CH); Kristina Bardon, Waldshut (DE)

Correspondence Address: JoAnn Villamizar;Ciba Corporation/Patent Department, 540 White Plains Road, P.O. Box 2005, Tarrytown, NY, 10591, US

(73) Assignee: CIBA SPECIALTY CHEMICALS HOLDING INC., Basel (CH)

(21) Appl. No.: 11/587,691

(22) PCT Filed: Apr. 20, 2005
(86) PCT No.: PCT/EP05/51731

§ 371 Date: Oct. 26, 2006

(30) Foreign Application Priority Data
Apr. 29, 2004 (EP) 04101826.8

(51) Int. Cl. B32B 9/00; C07D 239/24; C09K 11/00; C07D 251/12

(52) U.S. Cl. 428/690; 544/242; 544/180

(57) ABSTRACT

Disclosed are electroluminescent devices that comprise organic layers that contain triazine, or pyrimidine compounds. The compounds are suitable components of, for example, blueemitting, durable, organo-electroluminescent layers. The electroluminescent devices may be employed for full color display panels in, for example, mobile phones, televisions and personal computer screens.

The present invention relates to organo-electroluminescent (EL) devices, in particular EL devices that comprise durable, especially blue-emitting organo-electroluminescent layers. The organo-electroluminescent layers comprise certain triazine, or pyrimidine compounds.

The present invention is aimed at an electroluminescent device comprising an organic light-emitting layer that contains at least one blue-emitting triazine, or pyrimidine compound.

U.S. Pat. No. 6,352,791 relates to an electroluminescent arrangement, comprising at least two electrodes, and a light emitting layer system including at least one emitter layer and at least one electron-conducting layer, wherein the at least one electron-conducting layer does not emit light and includes one triazine compound, such as, for example,

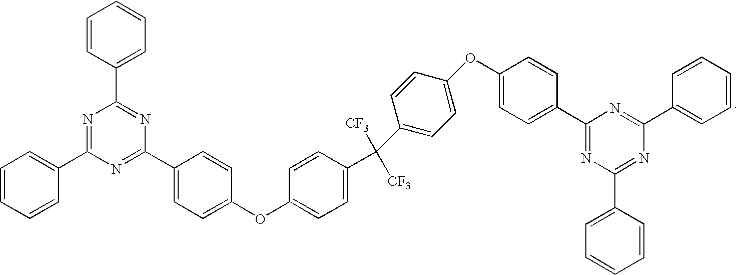

U.S. Pat. No. 6,225,467 is directed to organic electroluminescent (EL) devices, which contain an electron transport component comprised of triazine compounds, such as, for example, 4,6-tris(4-biphenylyl)-1,3,5-triazine, 2,4,6-tris[4-(4′-methylbiphenylyl)]—1,3,5-triazine, 2,4,6-tris[4-(4′-tert-butylbiphenylyl)-1,3,5-triazine, 2,4,6-tris[4-(3′,4′-dimethylbiphenylyl)]-1,3,5-triazine, 2,4,6-tris[4-(4′-methoxybiphenylyl)]-1,3,5-triazine, 2,4,6-tris[4-(3′-methoxybiphenylyl)]-1,3,5-triazine, 2,4-bis(4-biphenylyl)-6-phenyl-1,3,5-triazine and 2,4-bis(4-biphenylyl)-6-m-tolyl-1,3,5-triazine.

EP-A-1,202,608 relates to an electroluminescent arrangement, wherein a host material constituting the hole transporting layer is a compound of formula

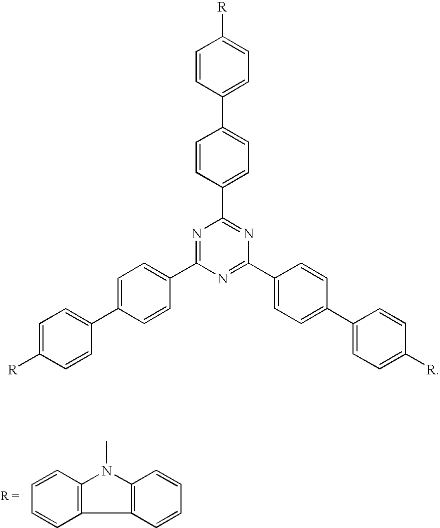

EP-A-1,013,740 relates to an electroluminescent element, wherein among others the following compound can be used as EL material:

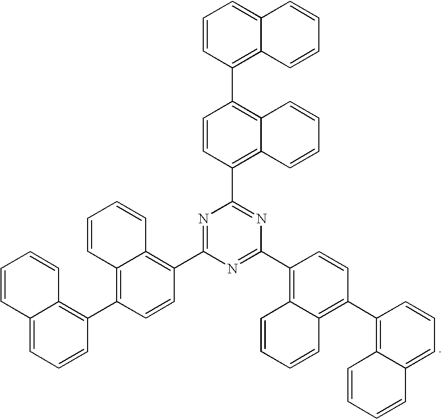

JP2003040873 relates to novel quinoxaline compounds, such as

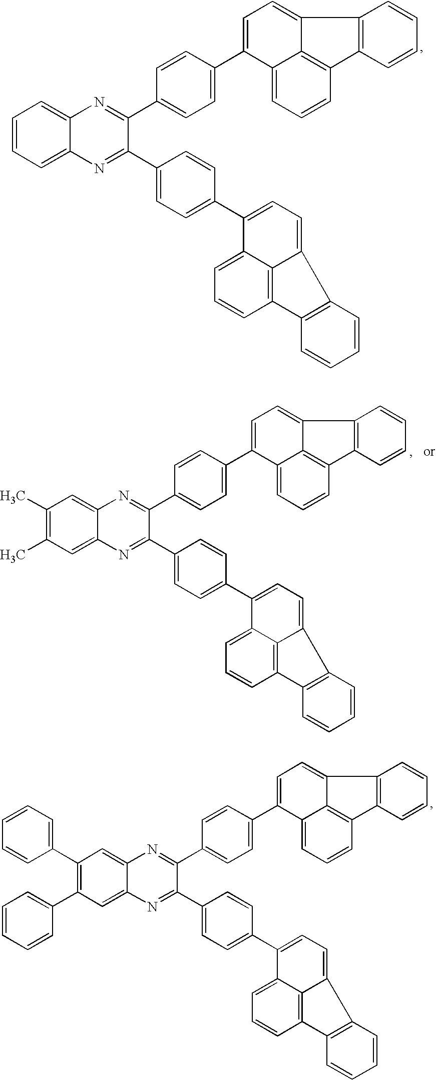

and their use in organic EL elements.

U.S. Pat. No. 5,104,740 teaches an electroluminescent element that comprises a fluorescent layer containing a coumarinic or azacoumarinic derivative and a hole transport layer, both made of organic compounds and laminated on top of the other.

U.S. Pat. No. 6,280,859 discloses certain polyaromatic organic compounds for use as a light-emitting material in organo-electroluminescent devices.

U.S. Pat. No. 5,116,708 is aimed at a hole transport material for EL devices.

WO98/04007 and EP-A-1013740 relate to an electroluminescent arrangement with the electron-conducting layer containing one or more compounds comprising triazine as basic substance.

EP-A-1013740 discloses the use of triazine compounds in EL devices.

EP-A-1,202,608 discloses EL devices comprising a carbazole compound of formula

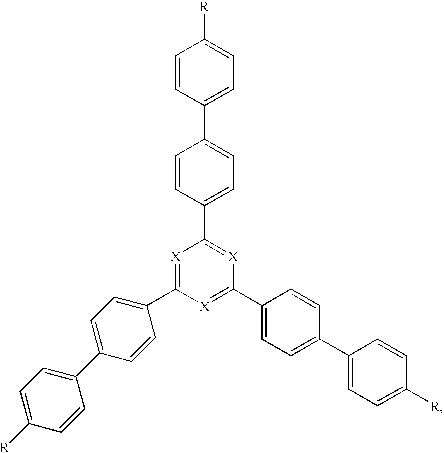

wherein R is

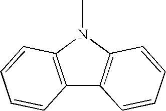

and X is C or N, which constitutes the hole transporting layer.

JP2002324678 relates to light emitting elements comprising at least one kind of compound of formula

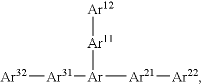

wherein
Ar¹¹, Ar²¹ and Ar³¹ denote arylene groups, Ar¹², Ar²² and Ar³² denote substituents or hydrogen atoms, wherein at least one of Ar¹¹, Ar²¹, Ar³¹, Ar¹², Ar²² and Ar³² is either a condensed ring aryl structure or a condensed ring heteroaryl structure; Ar denotes an arylene group or a heteroarylene group; and at least one amine derivative having a condensed ring group with two or more rings are contained in a luminous layer. As examples of compounds of the above formula, wherein Ar denotes a heteroarylene group the following two compounds are explicitly mentioned:

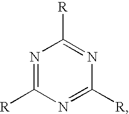

R is a group of formula

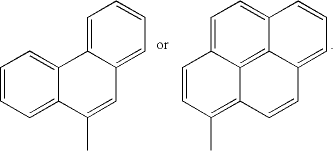

WO02/02714 relates to electroluminescent iridium compounds with fluorinated phenylpyridines, phenylpyrimidines, and phenylquinolines and devices made with such compounds.

U.S. Pat. No. 5,770,108 describes liquid crystal compositions comprising pyrimidine compounds of the following formula

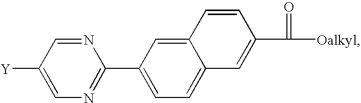

wherein Y is alkyl or —O-alkyl and liquid crystal element comprising said composition.

WO01/05863 relates to EL devices comprising arylamine-substituted poly(arylene vinylenes).

JP2000347432 describes the use of

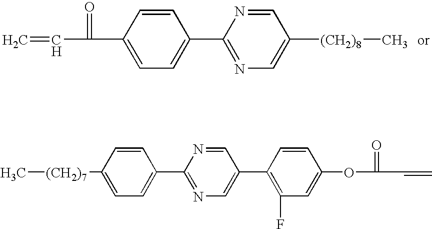

in EL devices.

EP-A-926216 relates to EL devices using triaryl amine compounds, such as

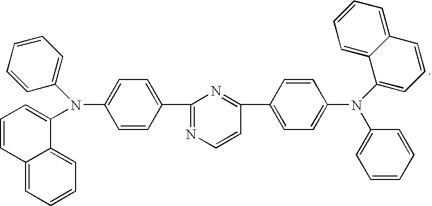

EP-A-690 053 relates to the use of conjugated compounds containing two or more pyrimidine rings, which are part of the conjugated system, as electroluminescent materials. The conjugated compounds described in EP-A-690 053 comprise pyrimidin-2,5-diyl groups which do not carry substituents at positions 4 and 6.

EP-A-563009 relates to EL devices comprising

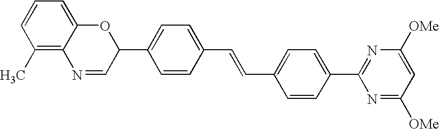

as light emitting material.

U.S. Pat. No. 5,077,142 relates to EL devices comprising a number of organic compounds as light emitting material. A pyrimidine moiety,

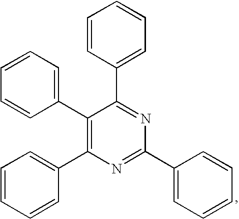

is listed among a long list of possible organic compounds.

It is the object of the present invention to provide a light emitting element with excellent light emitting characteristics and durability.

Accordingly, the present invention relates to an electroluminescent device comprising an anode, a cathode and one or a plurality of organic compound layers sandwiched therebetween, in which said organic compound layers comprise a compound of formula

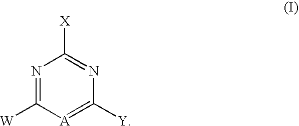

Preferably, the compound or compounds of the present invention emit light below about 520 nm, in particular between about 380 nm and about 520 nm.

The compound or compounds of the present invention have especially a NTSC coordinate of between about (0.12, 0.05) and about (0.16, 0.10), very especially a NTSC coordinate of about (0.14, 0.08).

The compound or compounds of the present invention have a melting point above about 150° C., preferably above about 200° C. and most preferred above about 250° C.

To obtain organic layers of this invention with the proper T_{g}, or glass transition temperature, it is advantageous that the present organic compounds have a glass transition temperature greater than about 100° C., for example greater than about 110° C., for example greater than about 120° C., for instance greater than about 130° C.

Accordingly, the present invention is directed to compounds of formula

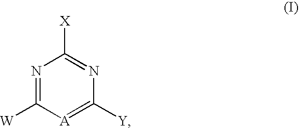

wherein

### A is CH, or N,

X is a group of the formula —(X¹)ₘ—(X²)ₙ—X³
W is a group of the formula —(W¹)ₐ—(W²)_{b}—W³, and
Y is a group of the formula —(Y¹)_{c}—(Y²)_{d}—Y³, wherein
a, b, c, d, m and n are independently of each other 0, or 1,
W¹, W², X¹, X², Y¹ and Y² are independently of each other a group of formula

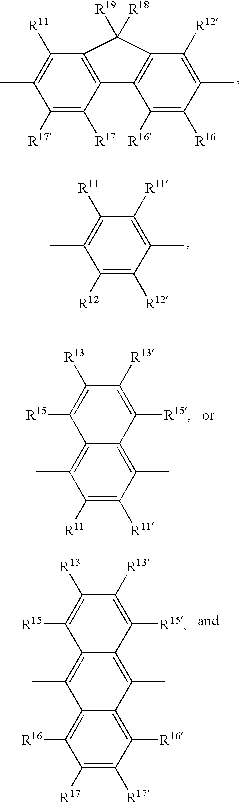

W³, X³ and Y³ are independently of each other a group of formula

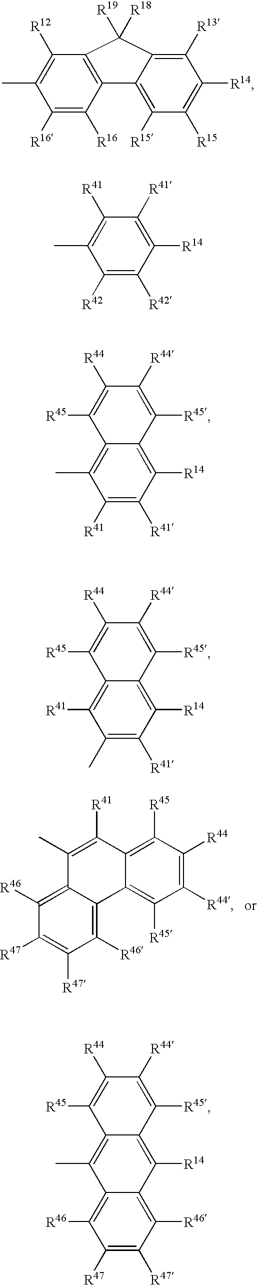

or a C₁₆-C₃₀aryl group, such as fluoranthenyl, triphenlenyl, chrysenyl, naphthacenyl, picenyl, perylenyl, pentaphenyl, hexacenyl, or pyrenyl, which can be substituted by G;
R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by G; C₁-C₁₈alkyl; C₁-C₁₈alkyl which is substituted by E and/or interrupted by D; C₁-C₁₈alkoxy; or C₁-C₁₈alkoxy which is substituted by E and/or interrupted by D; C₇-C₁₈aralkyl; or C₇-C₁₈aralkyl which is substituted by G;
R¹⁴ is H, C₁-C₁₈alkyl; or C₁-C₁₈alkyl which is substituted by E and/or interrupted by D; C₁-C₁₈alkoxy; or C₁-C₁₈alkoxy which is substituted by E and/or interrupted by D;

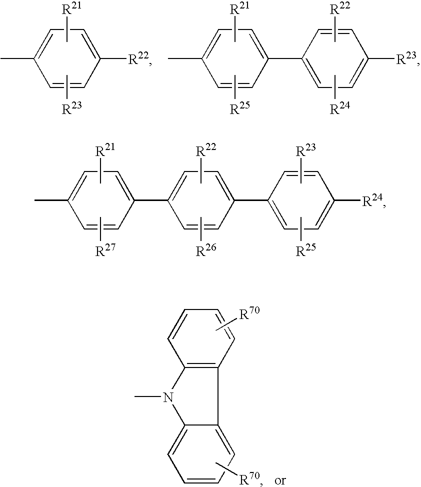

—NAr¹Ar², wherein Ar¹ and Ar² are independently of each other

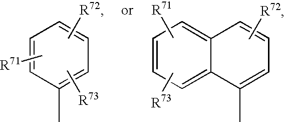

R²¹, R²², R²³, R²⁴, R²⁵, R²⁶ and R²⁷ are independently of each other H, E, C₁-C₁₈alkyl; C₁-C₁₈alkyl which is substituted by E and/or interrupted by D; C₇-C₁₈aralkyl; C₇-C₁₈aralkyl which is substituted by G;
R⁷⁰, R⁷¹, R⁷² and R⁷³ are independently of each other C₁-C₁₈alkyl, C₁-C₁₈alkyl which is interrupted by —O—, C₆-C₁₈aryl; C₆-C₁₈aryl which is substituted by C₁-C₁₈alkyl, —OR⁵, or —SR⁵, R¹⁸ and R¹⁹ are independently of each other C₁-C₁₈alkyl; C₁-C₁₈alkoxy, C₆-C₁₈aryl; C₇-C₁₈aralkyl; or R¹⁸ and R¹⁹ together form a ring especially a five- or six-membered ring, which can optionally be substituted by C₁-C₈alkyl,
D is —CO—; —COO—; —OCOO—; —S—; —SO—; —SO₂—; —O—; —NR⁵—; SiR⁶¹R⁶²—; —POR⁵—; —CR⁶³═CR⁶⁴; or —C≡C—;
E is —OR⁵; —SR⁵; —NR⁵R⁶; —COR⁸; —COOR⁷; —OCOOR⁷, —CONR⁵R⁶; —CN; or halogen;
G is E, or C₁-C₁₈alkyl,
wherein R⁵ and R⁶ are independently of each other C₆-C₁₈aryl; C₆-C₁₈aryl which is substituted by C₁-C₁₈alkyl, or C₁-C₁₈alkoxy; C₁-C₁₈alkyl or C₁-C₁₈alkyl which is interrupted by —O—; or

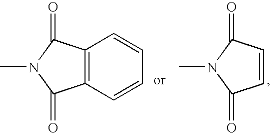

R⁵ and R⁶ together form a five or six membered ring, in particular
R⁷ is C₇-C₁₂alkylaryl; C₁-C₁₈alkyl; or C₁-C₁₈alkyl which is interrupted by —O—;
R⁸ is C₆-C₁₈aryl; C₆-C₁₈aryl which is substituted by C₁-C₁₈alkyl, or C₁-C₁₈alkoxy; C₁-C₁₈alkyl; C₇-C₁₂alkylaryl, or C₁-C₁₈alkyl which is interrupted by —O—;
R⁶¹ and R⁶² are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, C₁-C₁₈alkoxy; or C₁-C₁₈alkyl which is interrupted by —O—.

If W³ and/or Y³ are different from a C₁₆-C₃₀aryl group, X is preferably a group of the formula —X¹—X³, especially phenyl, or biphenyl.

If W³ and/or Y³ are a C₁₆-C₃₀aryl group, they are especially a fluoranthenyl, triphenlenyl,

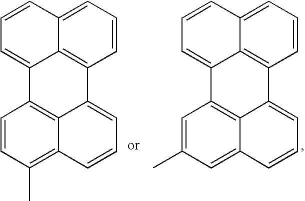

chrysenyl, naphthacen, picenyl, perylenyl, such as pentaphenyl, hexacenyl, or pyrenyl group, which can be substituted by G; very especially a fluoranthenyl group, which can be substituted by G.

In a preferred embodiment the present invention is directed to triazine compounds of formula

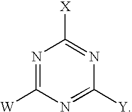

In a preferred embodiment the present invention is directed to pyrimidine compounds of formula

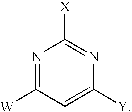

If W³, Y³ and/or X³ are different from a C₁₆-C₃₀aryl group, they are in one embodiment of the present application especially a C₆-C₁₀-aryl group, such as phenyl, 1-naphthyl, 2-naphthyl, 3- or 4-biphenyl, 9-phenanthryl, 2- or 9-fluorenyl, which is optionally substituted by C₁-C₆-alkyl, or C₁-C₄-alkoxy, especially

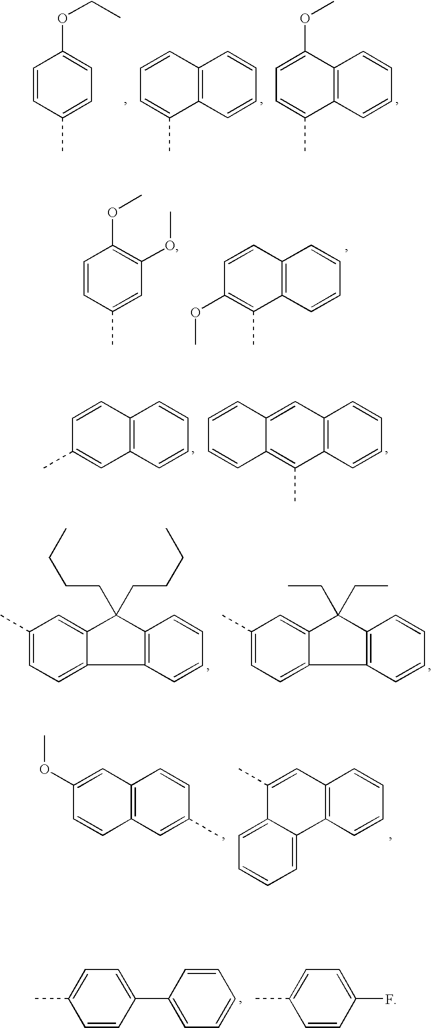

Preferably, R¹⁸ and R¹⁹ are independently of each other H, C₁-C₁₈alkyl, such as n-butyl, sec-butyl, hexyl, octyl, or 2-ethyl-hexyl, C₁-C₁₈alkyl which is substituted by E and/or interrupted by D, such as —CH₂(OCH₂CH₂)_{w}OCH₃, w=1, 2, 3, or 4, C₆-C₂₄aryl, such as phenyl, naphthyl, or biphenyl, C₆-C₂₄aryl which is substituted by G, such as —C₆H₄OCH₃, —C₆H₄OCH₂CH₃, —C₆H₃(OCH₃)₂, —C₆H₃(OCH₂CH₃)₂, —C₆H₄CH₃, —C₆H₃(CH₃)₂, —C₆H₂(CH₃)₃, or —C₆H₄tBu, or R¹⁸ and R¹⁹ together form a 5 or 6 membered ring, such as cyclohexyl, or cyclopentyl, which can optionally be substituted by C₁-C₈alkyl.

D is preferably —CO—, —COO—, —S—, —SO—, —SO₂—, —O—, —NR⁵—, wherein R⁵ is C₁-C₁₈alkyl, such as methyl, ethyl, n-propyl, iso-propyl, n-butyl, isobutyl, or sec-butyl, or C₆-C₂₄aryl, such as phenyl, naphthyl, or biphenyl.

E is preferably —OR⁵; —SR⁵; —NR⁵R⁶; —COR⁸; —COOR⁷; —CONR⁵R⁶; or —CN; wherein R⁵, R⁵, R⁷ and R⁸ are independently of each other C₁-C₁₈ alkyl, such as methyl, ethyl, n-propyl, isopropyl, n-butyl, isobutyl, sec-butyl, hexyl, octyl, or 2-ethyl-hexyl, or C₆-C₂₄aryl, such as phenyl, naphthyl, or biphenyl, or R⁵ and R⁶ together form a ring

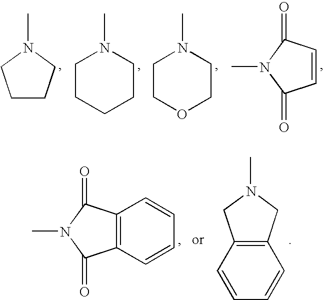

In a preferred embodiment of the present invention X is

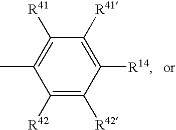

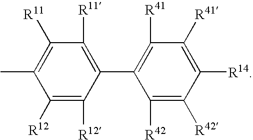

In another preferred embodiment of the present invention W and Y are a group of the formula —W¹—W²—W³. In another preferred embodiment of the present invention at least one of a and b and c and d is 1, or both a and b and c and d are 1.

R¹¹, R^{11′}, R¹², R^{12′}, R¹³, R^{13′}, R¹⁵, R^{15′}, R¹⁶, R^{16′}, R¹⁷ and R^{17′}, R⁴¹, R^{41′}, R⁴², R^{42′}, R⁴⁴, R_{44′}, R⁴⁵, R^{45′}, R⁴⁶, R^{46′}, R⁴⁷, and R^{47′} as well as R¹⁴ are preferably independently of each other H, E; or C₁-C₁₈alkyl; such as methyl, ethyl, n-propyl, iso-propyl, n-butyl, isobutyl, sec-butyl, t-butyl, 2-methylbutyl, n-pentyl, isopentyl, n-hexyl, 2-ethylhexyl, or n-heptyl, C₁-C₁₈alkyl which is substituted by E and/or interrupted by D, such as —CH₂OCH₃, —CH₂OCH₂CH₃, —CH₂OCH₂CH₂OCH₃, or —CH₂OCH₂CH₂OCH₂CH₃, C₆-C₂₄aryl, such as phenyl, naphthyl, or biphenyl, C₆-C₂₄aryl which is substituted by G, such as —C₆H₄OCH₃, —C₆H₄OCH₂CH₃; wherein

D is —O—, E is —OR⁵; —SR⁵; —NR⁵R⁶; —COR⁸; —COOR⁷; —CONR⁵R⁶; —CN; —OCOOR⁷; or halogen;
G is E, or C₁-C₈alkyl; wherein R⁵ and R⁶ are independently of each other C₆-C₁₂aryl, or C₁-C₈alkyl;
R⁷ is C₇-C₁₂alkylaryl, or C₁-C₈alkyl; and
R⁸ is C₆-C₁₂aryl; or C₁-C₈alkyl.

In a preferred embodiment the present invention is directed to compounds of formula

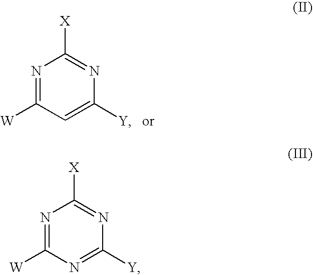

wherein

### X is

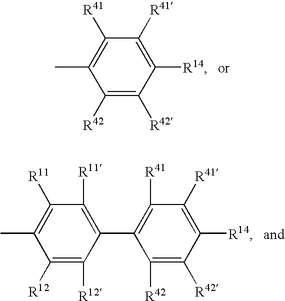

W and Y are a group of the formula —W¹—W²—W³, wherein
W¹ is a group of formula

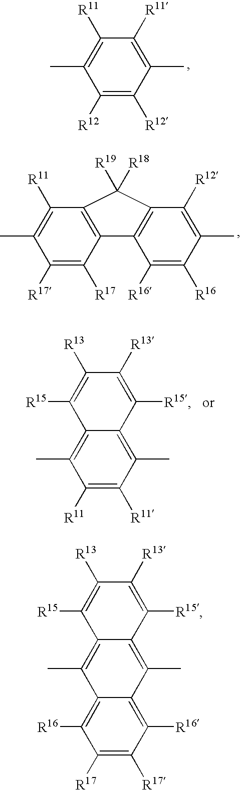

W² is a group of formula
W³ is a group of formula

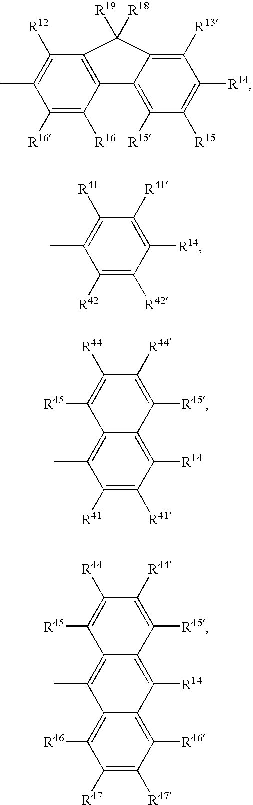

wherein
R¹¹, R^{11′}, R¹², R^{12′}, R¹³, R^{13′}, R¹⁴, R¹⁵, R^{15′}, R¹⁶, R^{16′}, R¹⁷, R^{17′}, R¹⁸, R¹⁹, R⁴¹. R^{41′}, R⁴², R^{42′}, R⁴⁴, R^{44′}, R⁴⁵, R^{45′}, R⁴⁶, R^{46′}, R⁴⁷ and R^{47′} are as defined above, or R^{15′} and R⁴¹ or R^{15′} and R⁴⁵ represents a single carbon carbon bond, or X, W and Y are a group of the formula —W¹—W²—W³, wherein W¹, W² and W³ are as defined above.

In said embodiment compounds of formula II, or III are especially preferred, wherein

### X is

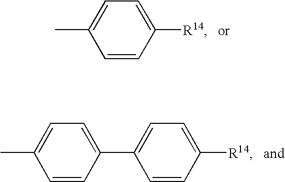

W and Y are a group of the formula —W¹—W²—W³, wherein
W¹ is a group of formula

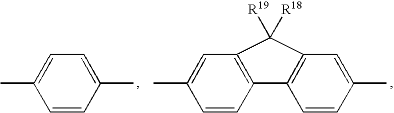

W² is a group of formula

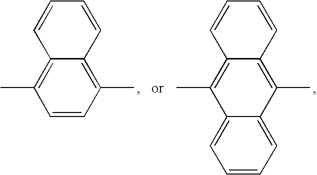

W³ is a group of formula

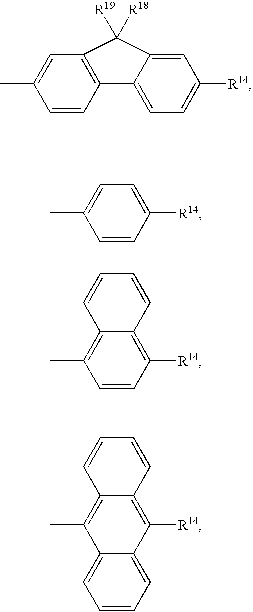

### R¹⁴

wherein
R¹⁴ is H, C₁-C₈alkyl, or C₁-C₈alkoxy, and
wherein R¹⁸ and R¹⁹ are independently of each other C₁-C₈alkyl, or cyclohexyl, wherein the following compounds are excluded:

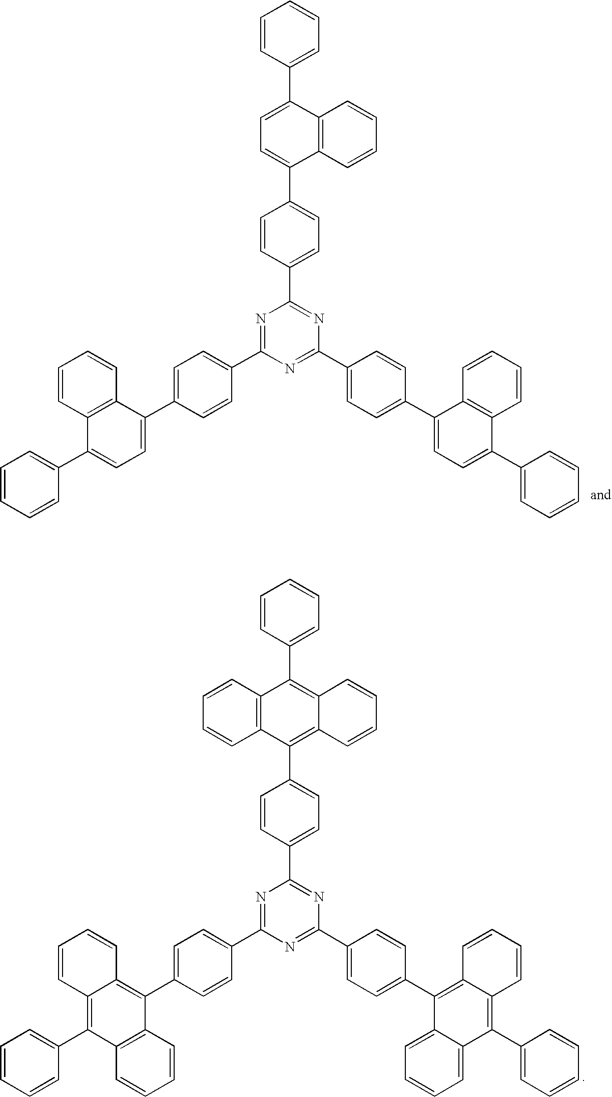

In a further preferred embodiment the present invention is directed to compounds of formula

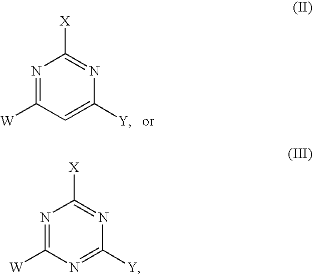

wherein

### X is

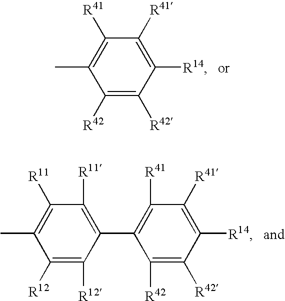

W and Y are a group Ar¹—Ar², wherein
Ar¹ is a group of formula

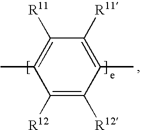

Ar² is a group of formula

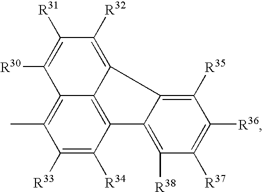

wherein
R³⁰, R³¹, R³², R³³, R³⁴, R³⁵, R³⁶, R³⁷ and R³⁸ are independently of each other H, E, C₆-C₁₈aryl; C₆-C₁₈aryl which is substituted by G; C₁-C₁₈alkyl; C₁-C₁₈alkyl which is substituted by E and/or interrupted by D; C₇-C₁₈aralkyl; or C₇-C₁₈aralkyl which is substituted by G;
e is an integer 1, or 2, or
X, W and Y are independently of each other a group Ar¹—Ar², wherein Ar¹ and Ar² are as defined above, and
D, E, G, R¹¹, R^{11′}, R¹², R^{12′}, R⁴¹, R^{41′}, R⁴², R^{42′}, and R¹⁴ are defined above.

If X, W and Y are independently of each other a group Ar¹—Ar², they can be different, but they have preferably the same meaning.

In said embodiment compounds of formula II, or III are especially preferred, wherein

### X is

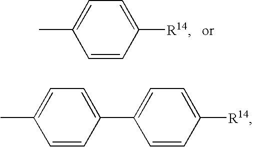

and
W and Y are a group Ar¹—Ar², wherein
Ar¹ is a group of formula

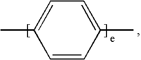

Ar² is a group of formula

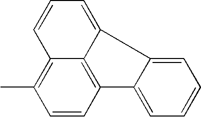

e is an integer 1, or 2,
R¹⁴ is H, C₁-C₈alkyl, or C₁-C₈alkoxy, or
X, W and Y are a group Ar¹—Ar², wherein Ar¹ and Ar² are as defined above.

In a further preferred embodiment the present invention is directed to compounds of formula

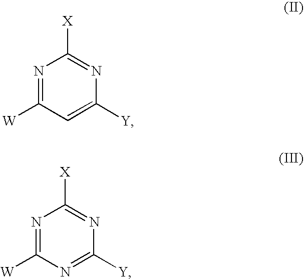

wherein

### X is

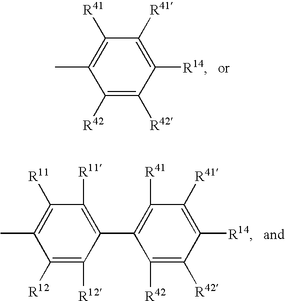

W and Y are a group of the formula —W¹—(W²)_{b}—W³, wherein b is 1, or 2,
W¹ and W² are independently of each other a group of formula

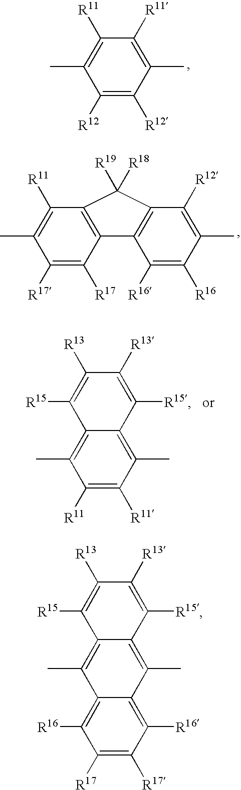

W³ is a group of formula

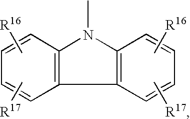

or —NR⁵⁰R⁵¹, wherein R⁵⁰ and R⁵¹ are
independently of each other a group of formula

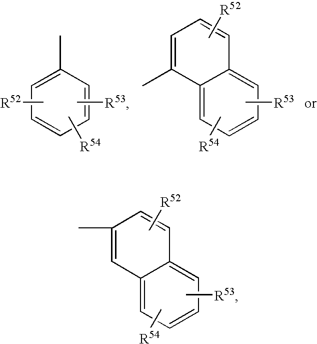

wherein R⁵², R⁵³ and R⁵⁴ are independently of each other hydrogen, C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group or a siloxanyl group, wherein R¹¹, R^{11′}, R¹², R^{12′}, R¹³, R^{13′}, R¹⁴, R¹⁵, R^{15′}, R¹⁶, R^{16′}, R¹⁷, R^{17′}, R¹⁸, R¹⁹, R⁴¹, R^{41′}, R⁴² and R^{42′} are as defined above, or X, W and Y are independently of each other a group of the formula —W¹—(W²)_{b}—W³, wherein b, W¹, W² and W³ are as defined above.

If X, W and Y are independently of each other a group —W¹—(W²)_{b}—W³, they can be different, but they have preferably the same meaning.

In said embodiment compounds of formula II, or III are especially preferred, wherein

### X is

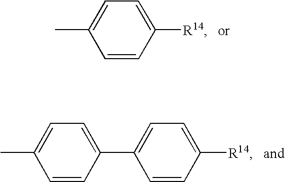

W and Y are a group of the formula —W¹—(W²)_{b}—W³, wherein b is 1, or 2,
W¹ is a group of formula

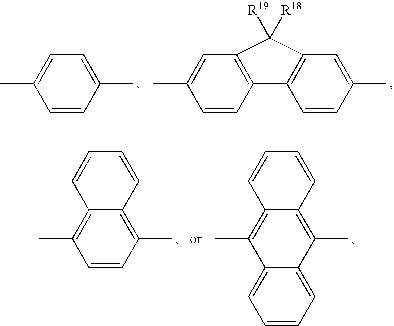

W² is a group of formula
W³ is a group of formula

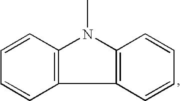

or —NR⁵⁰R⁵¹, wherein R⁵⁰ and R⁵¹ are independently of each other a group of formula

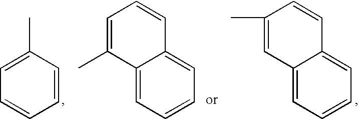

R¹⁴ is H, C₁-C₈alkyl, or C₁-C₈alkoxy, and
R¹⁸ and R¹⁹ are independently of each other C₁-C₈alkyl.

In an especially preferred embodiment the present invention is directed to compounds of formula

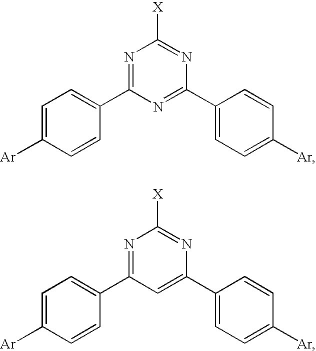

wherein X is a group of formula

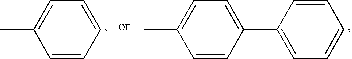

which can optionally be substituted by G, Ar is a group of formula

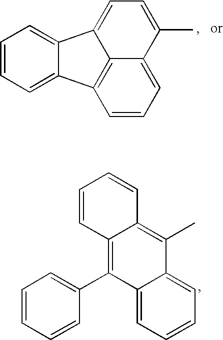

which can optionally be substituted by G, or X is a group of formula

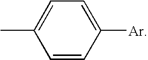

Specific examples of preferred triazine and pyrimidine compounds are:

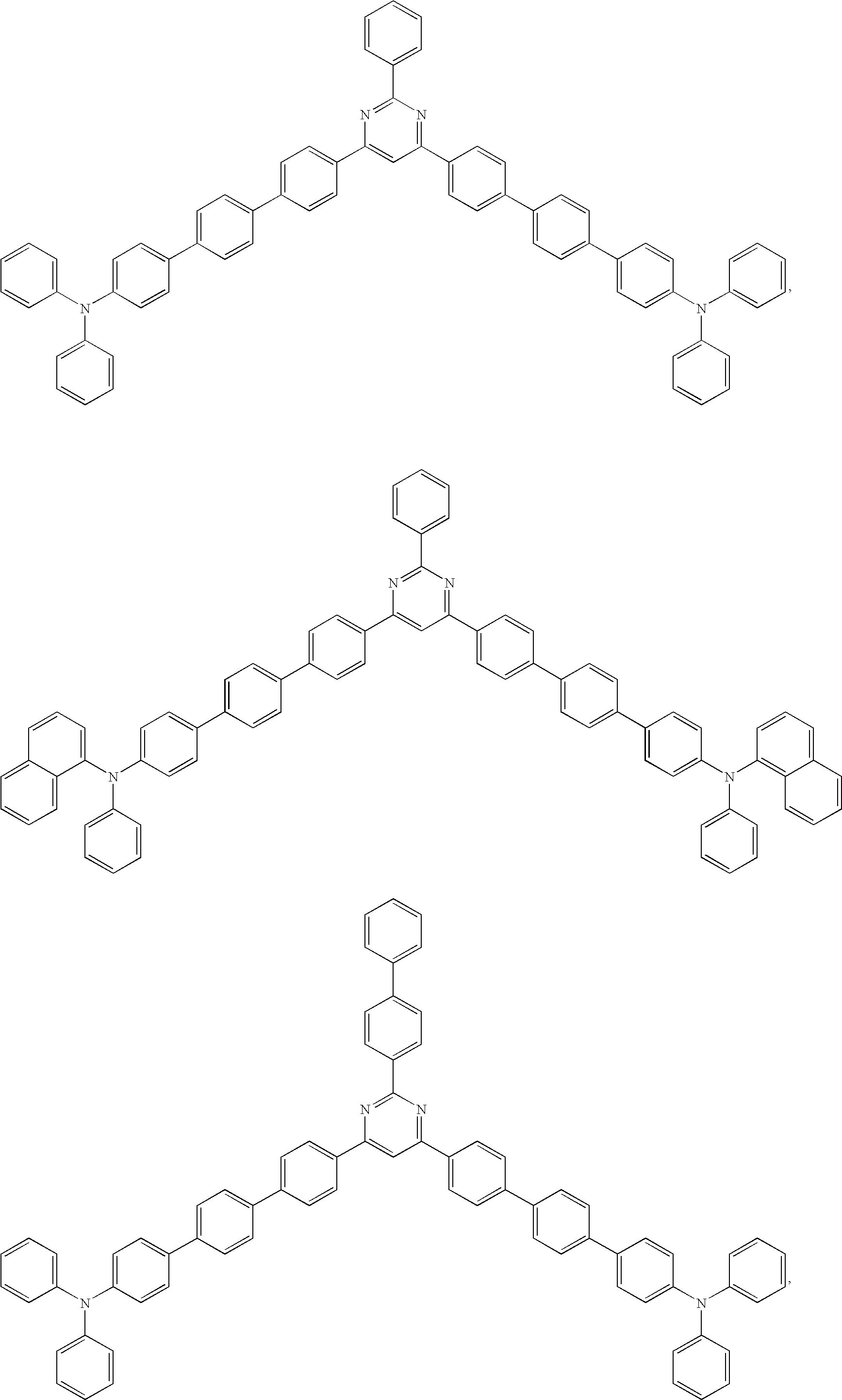

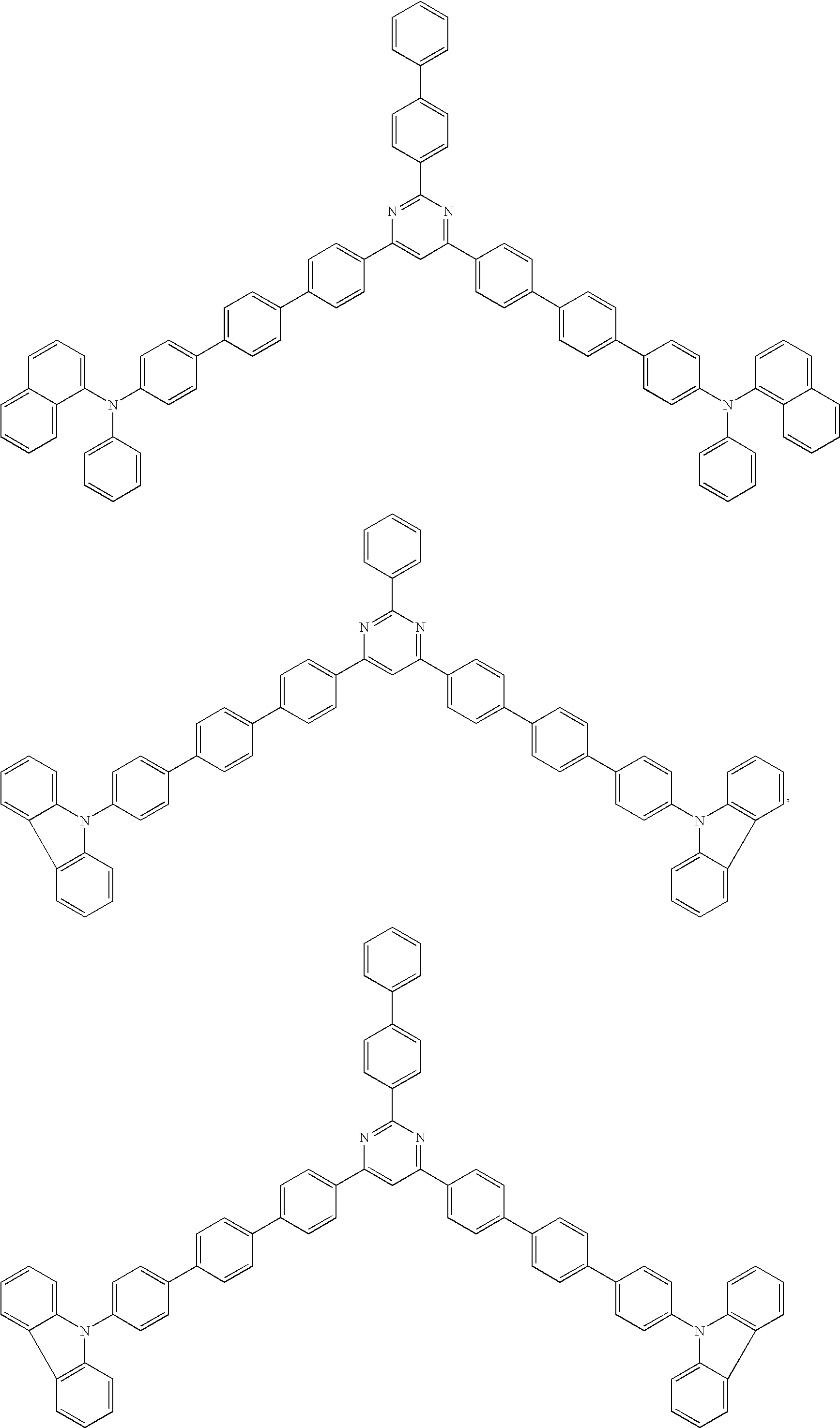

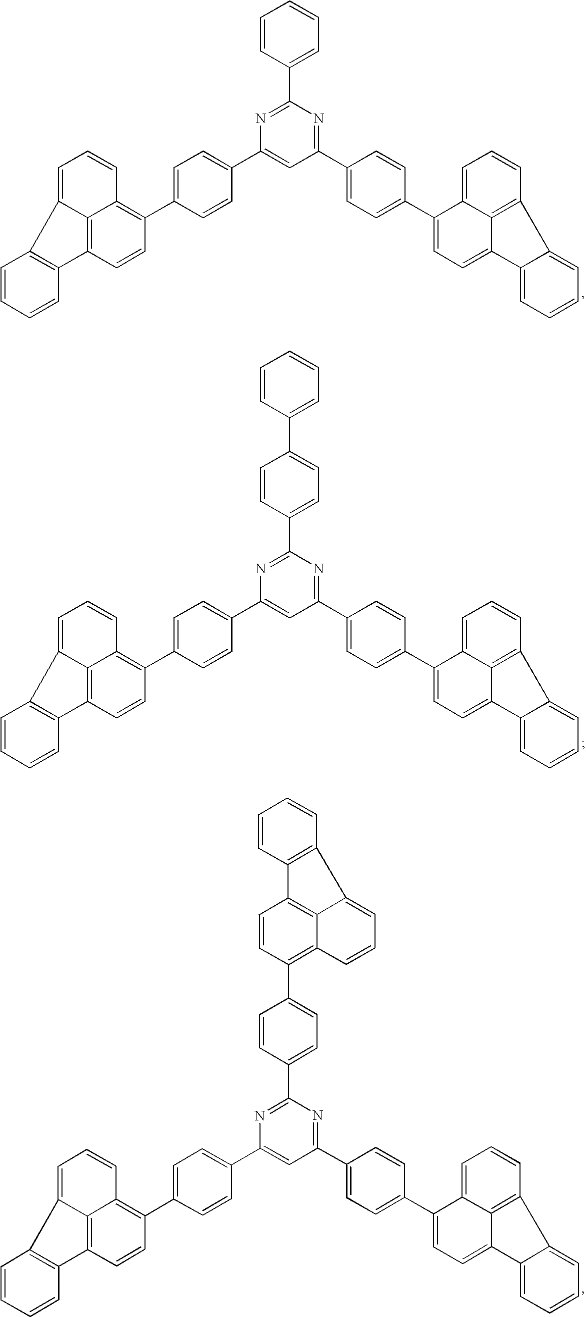

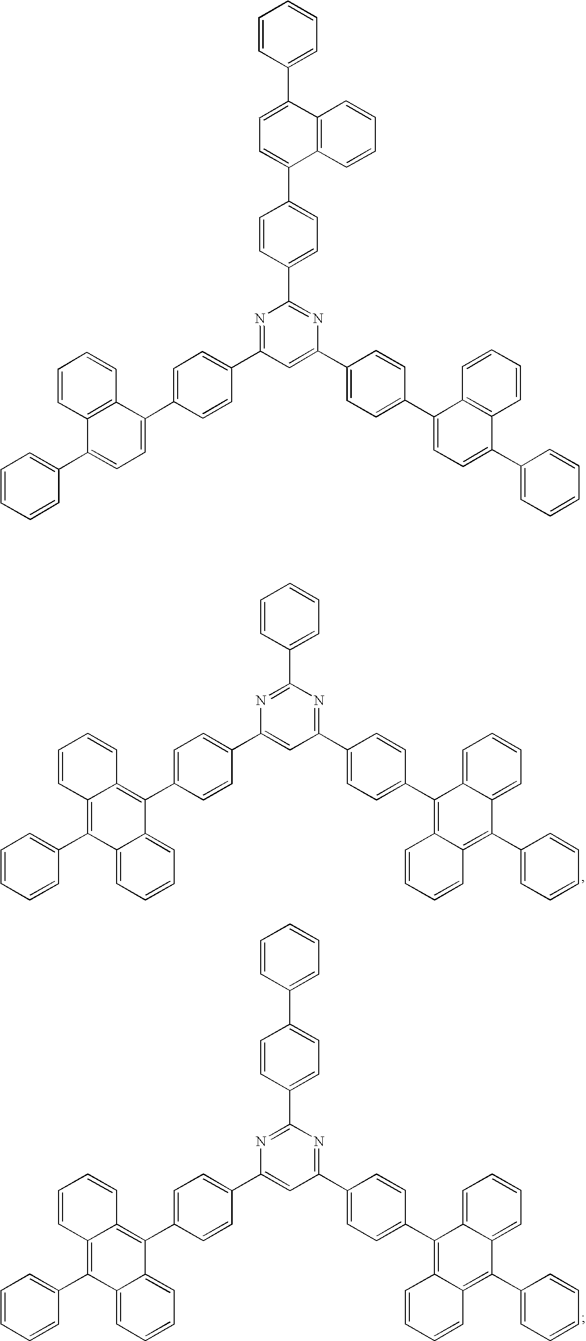

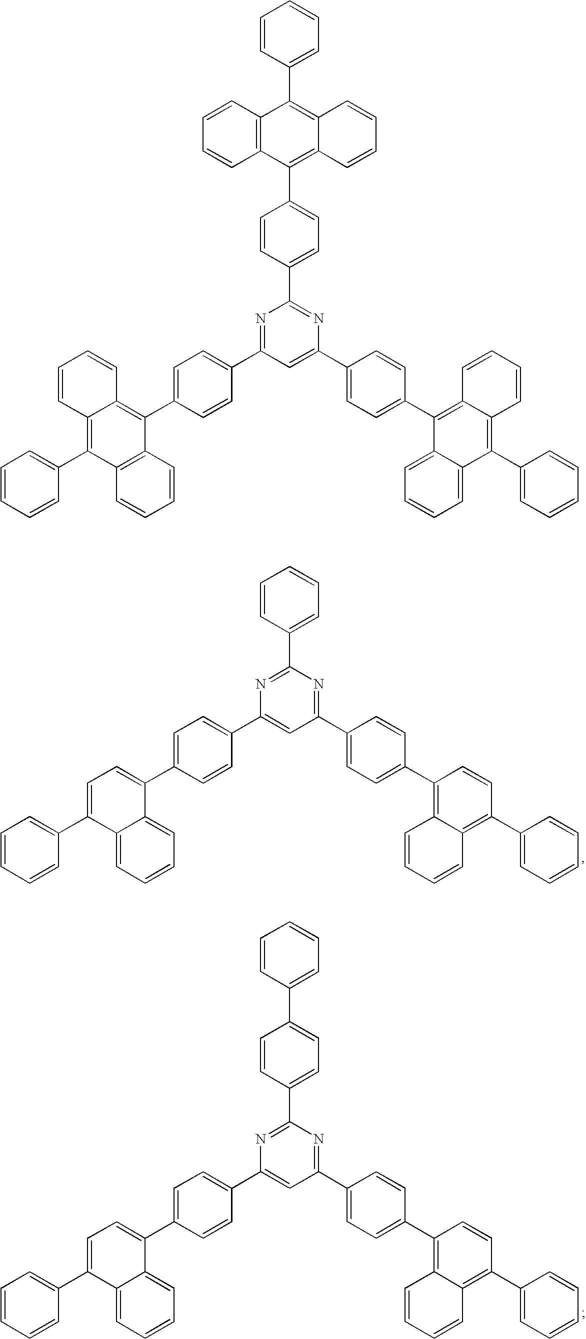

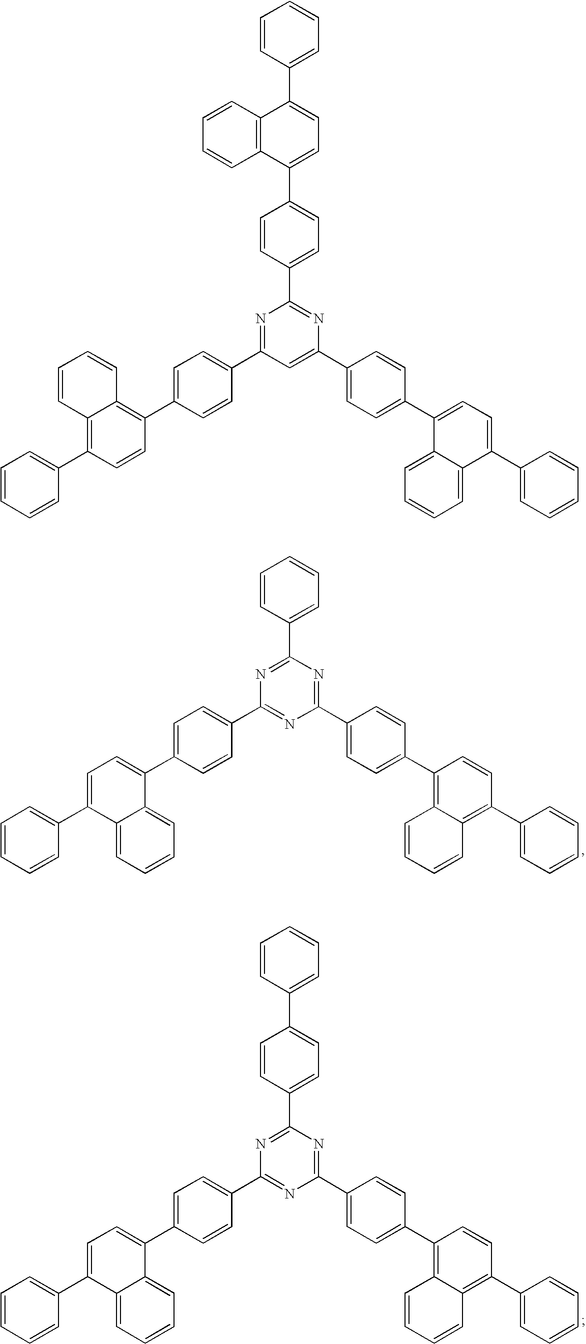

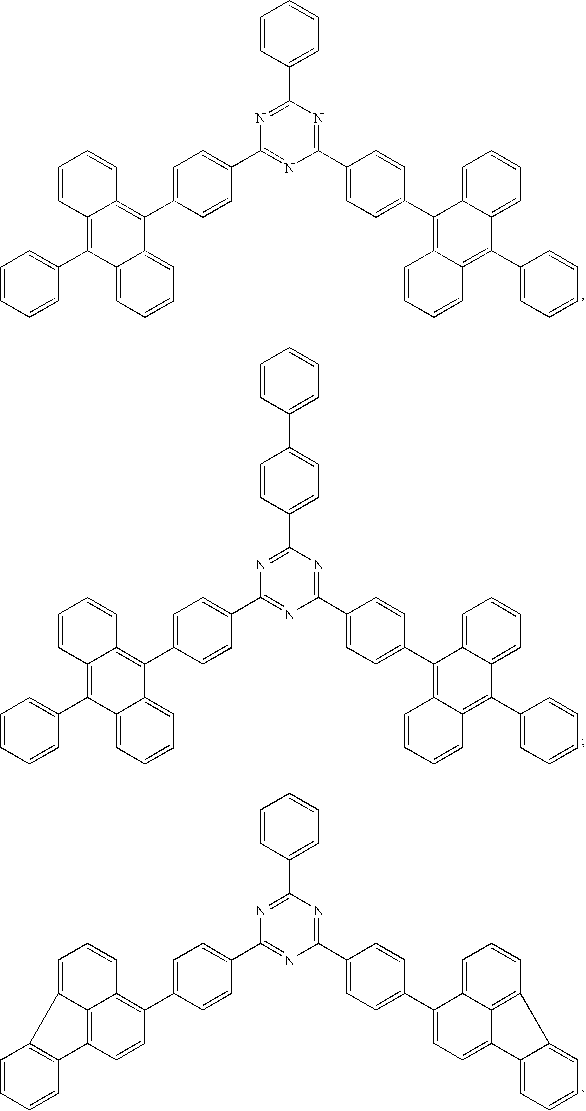

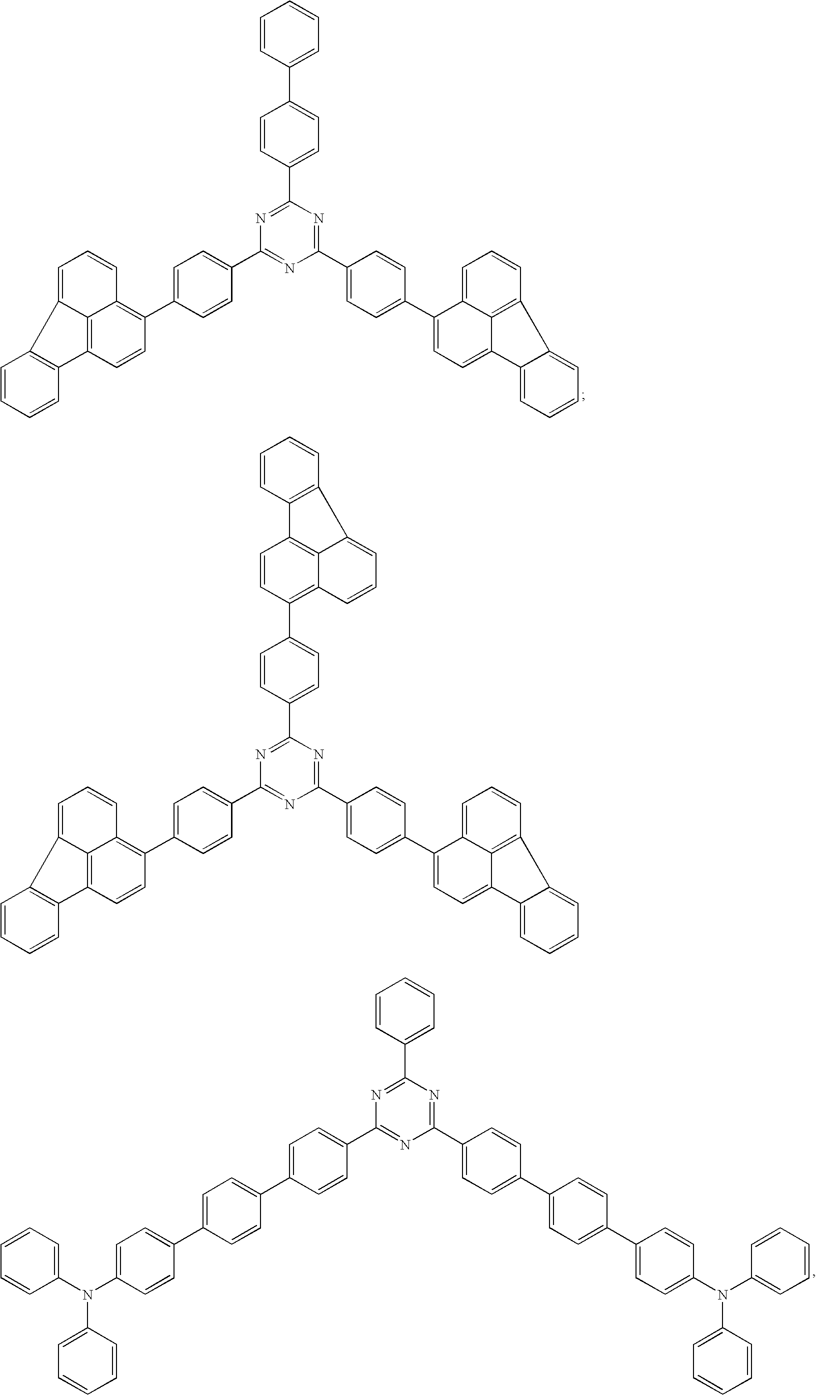

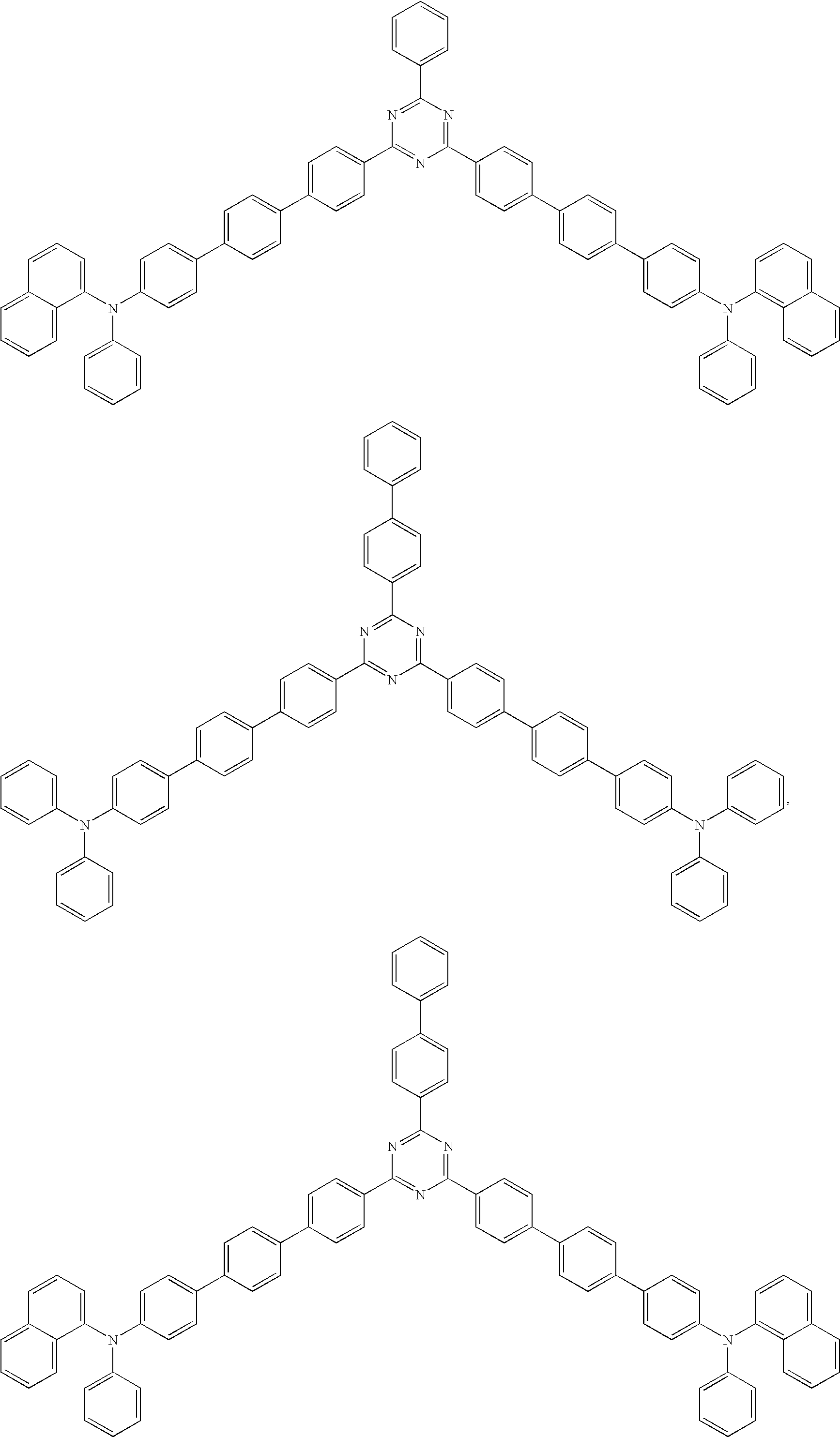

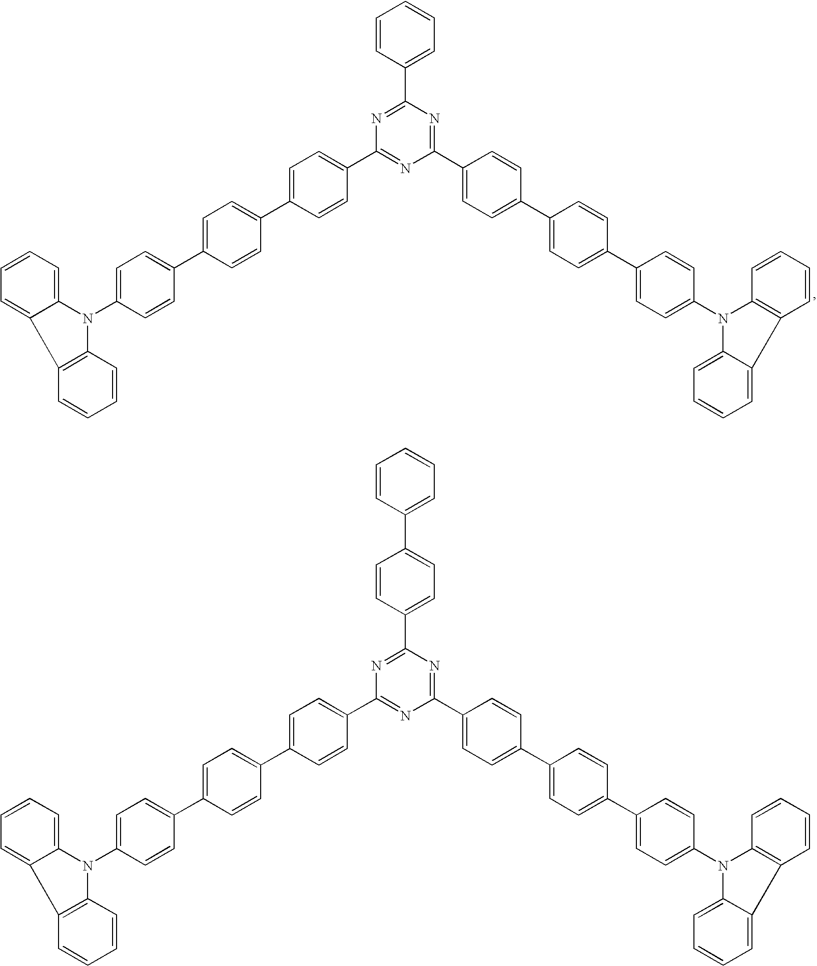

The present triazine and pyrimidine compounds show a high solid state fluorescence in the desired wavelength range and can be prepared according to or analogous to known procedures (se, for example, PCT/EP03/11637 and PCT/EP2004/050146).

The triazine compounds of the present invention of the formula:

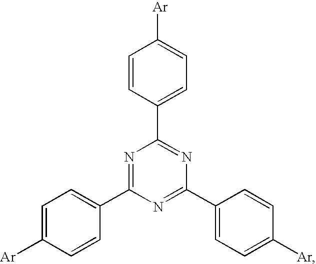

wherein Ar is W²—W³, can, for example, be prepared according to a process, which comprises reacting a derivative of formula

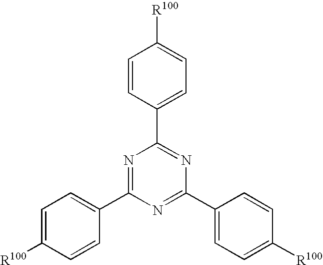

wherein R¹⁰⁰ stands for halogen such as chloro or bromo, preferably bromo, or E having the meaning of

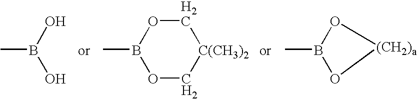

wherein a is 2 or 3,
with boronic acid derivative

### E-Ar,

or—in case R¹⁰⁰ is not halogen—

### Hal-Ar,

wherein Hal stands for halogen, preferably for bromo,
in the presence of an allylpalladium catalyst of the μ-halo(triisopropylphosphine)(η³-allyl)palladium(II) type (see for example WO99/47474).

Accordingly, unsymmetrical substituted triazine compounds of the present invention of the formula:

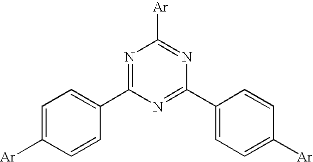

can, for example, be prepared according to a process, which comprises reacting a derivative of formula

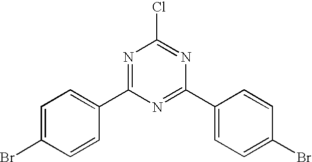

with a boronic acid derivative E-Ar, wherein E is as defined above.

The pyrimidine compounds of the present invention, comprising the following units:

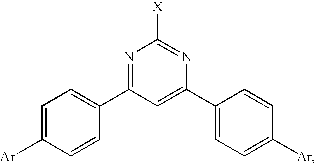

wherein X is

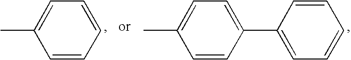

Ar is defined as above and is especially

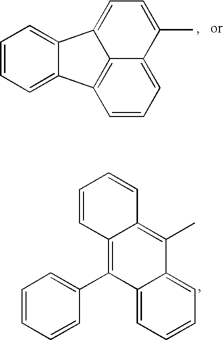

can be prepared according to a process, which comprises reacting a derivative of formula

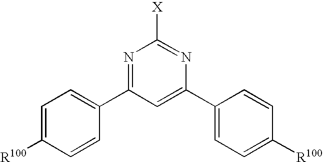

R¹⁰⁰ stands for halogen such as chloro or bromo, preferably bromo,
with boronic acid derivative
E-Ar, E having the meaning of

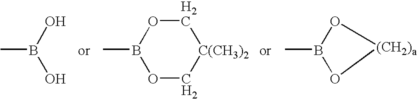

wherein a is 2 or 3,
in the presence of an allylpalladium catalyst of the μ-halo(triisopropylphosphine)(η³-allyl)palladium(II) type (see for example WO99/47474).

Preferably, the reaction is carried out in the presence of an organic solvent, such as an aromatic hydrocarbon or a usual polar organic solvent, such as benzene, toluene, xylene, tetrahydrofurane, or dioxane, or mixtures thereof, most preferred toluene. Usually, the amount of the solvent is chosen in the range of from 1 to 10 l per mol of boronic acid derivative. Also preferred, the reaction is carried out under an inert atmosphere such as nitrogen, or argon.

Further, it is preferred to carry out the reaction in the presence of an aqueous base, such as an alkali metal hydroxide or carbonate such as NaOH, KOH, Na₂CO₃, K₂CO₃, Cs₂CO₃ and the like, preferably an aqueous K₂CO₃ solution is chosen. Usually, the molar ratio of the base to compound III is chosen in the range of from 0.5:1 to 50:1.

Generally, the reaction temperature is chosen in the range of from 40 to 180° C., preferably under reflux conditions.

Preferred, the reaction time is chosen in the range of from 1 to 80 hours, more preferably from 20 to 72 hours.

In a preferred embodiment a usual catalyst for coupling reactions or for polycondensation reactions is used, preferably Pd-based catalyst such as known tetrakis(triarylphosphonium)-palladium, preferably (Ph₃P)₄Pd and derivatives thereof. Usually, the catalyst is added in a molar ratio from inventive DPP polymer to the catalyst in the range of from 100:1 to 10:1, preferably from 50:1 to 30:1.

Also preferred, the catalyst is added as in solution or suspension. Preferably, an appropriate organic solvent such as the ones described above, preferably benzene, toluene, xylene, THF, dioxane, more preferably toluene, or mixtures thereof, is used. The amount of solvent usually is chosen in the range of from 1 to 10 l per mol of boronic acid derivative. The obtained inventive polymer can be isolated by well-known methods. Preferably, after cooling down the reaction mixture to room temperature, it is poured into acetone and the obtained precipitation is filtered off, washed and dried.

C₁-C₁₈Alkyl is a branched or unbranched radical such as for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, or octadecyl.

C₁-C₁₈Alkoxy radicals are straight-chain or branched alkoxy radicals, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy.

C₂-C₁₈Alkenyl radicals are straight-chain or branched alkenyl radicals, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂₋₂₄Alkynyl is straight-chain or branched and preferably C₂₋₈alkynyl, which may be unsubstituted or substituted, such as, for example, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten-4-yn-1-yl, 1,3-hexadiyn-5-yl, 1-octyn-8-yl, 1-nonyn-9-yl, 1-decyn-10-yl or 1-tetracosyn-24-yl,

C₄-C₁₈cycloalkyl is preferably C₅-C₁₂cycloalkyl, such as, for example, cyclopentyl, cyclohexyl, cycloheptyl, cyclooctyl, cyclononyl, cyclodecyl, cyclododecyl. Cyclohexyl and cyclododecyl are most preferred.

The term “aryl group” is typically C₆-C₃₀aryl, such as phenyl, indenyl, azulenyl, naphthyl, biphenyl, terphenylyl or quadphenylyl, as-indacenyl, s-indacenyl, acenaphthylenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Examples of C₆-C₁₈aryl are phenyl, 1-naphthyl, 2-naphthyl, 3- or 4-biphenyl, 9-phenanthryl, 2- or 9-fluorenyl, which may be unsubstituted or substituted.

C₇-C₂₄aralkyl radicals are preferably C₇-C₁₈aralkyl radicals, which may be substituted, such as, for example, benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl or ω-phenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted.

C₇-C₁₂alkylaryl is, for example, a phenyl group substituted with one, two or three C₁-C₆alkyl groups, such as, for example, 2-, 3-, or 4-methylphenyl, 2-, 3-, or 4-ethylphenyl, 3-, or 4-isopropylphenyl, 3,4-dimethylphenyl, 3,5-dimethyl phenyl, or 3,4,5-trimethylphenyl.

The term “heteroaryl group”, especially C₂-C₃₀heteroaryl, is a ring, wherein nitrogen, oxygen or sulfur are the possible hetero atoms, and is typically an unsaturated heterocyclic radical with five to 18 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocyclic radicals, which may be unsubstituted or substituted.

Halogen is fluorine, chlorine, bromine and iodine.

The terms “haloalkyl” mean groups given by partially or wholly substituting the above-mentioned alkyl group, with halogen, such as trifluoromethyl etc. The “aldehyde group, ketone group, ester group, carbamoyl group and amino group” include those substituted by an alkyl group, a cycloalkyl group; an aryl group, an aralkyl group or a heterocyclic group, wherein the alkyl group, the cycloalkyl group, the aryl group, the aralkyl group and the heterocyclic group may be unsubstituted or substituted. The term “silyl group” means a group of formula —SiR⁶²R⁶³R⁶⁴, wherein R⁶², R⁶³ and R⁶⁴ are independently of each other a C₁-C₈alkyl group, in particular a C₁-C₄ alkyl group, a C₆-C₂₄aryl group or a C₇-C₁₂aralkylgroup, such as a trimethylsilyl group. The term “siloxanyl group” means a group of formula —O—SiR⁶²R⁶³R⁶⁴, wherein R⁶², R⁶³ and R⁶⁴ are as defined above, such as a trimethylsiloxanyl group.

Examples of a five or six membered ring formed by R⁵ and R⁶ are heterocycloalkanes or heterocycloalkenes having from 3 to 5 carbon atoms which can have one additional hetero atom selected from nitrogen, oxygen and sulfur, for example

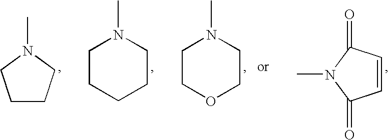

which can be part of a bicyclic system, for example

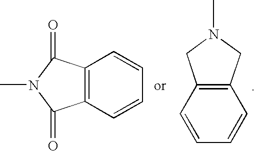

Possible substituents of the above-mentioned groups are C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group or a silyl group.

As described above, the aforementioned radicals may be substituted by E and/or, if desired, interrupted by D. Interruptions are of course possible only in the case of radicals containing at least 2 carbon atoms connected to one another by single bonds; C₆-C₁₈aryl is not interrupted; interrupted arylalkyl or alkylaryl contains the unit D in the alkyl moiety. C₁-C₁₈alkyl substituted by one or more E and/or interrupted by one or more units D is, for example, (CH₂CH₂O)ₙ—R^{x}, where n is a number from the range 1-9 and R^{x} is H or C₁-C₁₀alkyl or C₂-C₁₀alkanoyl (e.g. CO—CH(C₂H₅)C₄H₉), CH₂—CH(OR^{y}′)—CH₂—O—R^{y}, where R^{y} is C₁-C₁₈alkyl, C₅-C₁₂cycloalkyl, phenyl, C₇-C₁₅-phenylalkyl, and R^{y}′ embraces the same definitions as R^{y} or is H; C₁-C₈alkylene-COO—R^{z}, e.g. CH₂COOR^{z}, CH(CH₃)COOR^{z}, C(CH₃)₂COOR^{z}, where R^{z} is H, C₁-C₁₈alkyl, (CH₂CH₂O)₁₋₉—R^{x}, and R^{x} embraces the definitions indicated above; CH₂CH₂—O—CO—CH═CH₂; CH₂CH(OH)CH₂—O—CO—C(CH₃)═CH₂.

The electroluminescent devices may be employed for full color display panels in, for example, mobile phones, televisions and personal computer screens.

The electroluminescent devices of the present invention are otherwise designed as is known in the art, for example as described in U.S. Pat. Nos. 5,518,824, 6,225,467, 6,280,859, 5,629,389, 5,486,406, 5,104,740, 5,116,708 and 6,057,048, the relevant disclosures of which are hereby incorporated by reference.

For example, organic EL devices contain one or more layers such as: substrate; base electrode; hole-injecting layer; hole transporting layer; emitter layer; electron-transporting layer; electron-injecting layer; top electrode; contacts and encapsulation. This structure is a general case and may have additional layers or may be simplified by omitting layers so that one layer performs a plurality of tasks. For instance, the simplest organic EL device consists of two electrodes which sandwich an organic layer that performs all functions, including the function of light emission.

A preferred EL device comprises in this order:

(a) an anode,
(b) a hole injecting layer and/or a hole transporting layer,
(c) a light-emitting layer,
(d) optionally an electron transporting layer and
(e) a cathode.

In particular, the present organic compounds function as light emitters and are contained in the light emission layer or form the light-emitting layer.

The light emitting compounds of this invention exhibit intense fluorescence in the solid state and have excellent electric-field-applied light emission characteristics. Further, the light emitting compounds of this invention are excellent in the injection of holes from a metal electrode and the transportation of holes; as well as being excellent in the injection of electrons from a metal electrode and the transportation of electrons. They are effectively used as light emitting materials and may be used in combination with other hole transporting materials, other electron transporting materials or other dopants.

The organic compounds of the present invention form uniform thin films. The light emitting layers may therefore be formed of the present organic compounds alone.

Alternatively, the light-emitting layer may contain a known light-emitting material, a known dopant, a known hole transporting material or a known electron transporting material as required. In the organic EL device, a decrease in the brightness and life caused by quenching can be prevented by forming it as a multi-layered structure. The light-emitting material, a dopant, a hole-injecting material and an electron-injecting material may be used in combination as required. Further, a dopant can improve the light emission brightness and the light emission efficiency, and can attain the red or blue light emission. Further, each of the hole transporting zone, the light-emitting layer and the electron transporting zone may have the layer structure of at least two layers. In the hole transporting zone in this case, a layer to which holes are injected from an electrode is called “hole-injecting layer”, and a layer which receives holes from the hole-injecting layer and transport the holes to a light-emitting layer is called “hole transporting layer”. In the electron transporting zone, a layer to which electrons are injected from an electrode is called “electron-injecting layer”, and a layer which receives electrons from the electron-injecting layer and transports the electrons to a light-emitting layer is called “electron transporting layer”. These layers are selected and used depending upon factors such as the energy level and heat resistance of materials and adhesion to an organic layer or metal electrode.

The light-emitting material or the dopant which may be used in the light-emitting layer together with the organic compounds of the present invention includes for example anthracene, naphthalene, phenanthrene, pyrene, tetracene, coronene, chrysene, fluorescein, perylene, phthaloperylene, naphthaloperylene, perinone, phthaloperinone, naphthaloperinone, diphenylbutadiene, tetraphenylbutadiene, coumarine, oxadiazole, aldazine, bisbenzoxazoline, bisstyryl, pyrazine, cyclopentadiene, quinoline metal complex, aminoquinoline metal complex, benzoquinoline metal complex, imine, diphenylethylene, vinyl anthracene, diaminocarbazole, pyran, thiopyran, polymethine, merocyanine, an imidazole-chelated oxynoid compound, quinacridone, rubrene, and fluorescent dyestuffs for a dyestuff laser or for brightening.

The compounds of the present invention and the above compound or compounds that can be used in a light-emitting layer may be used in any mixing ratio for forming a light-emitting layer. That is, the organic compounds of the present invention may provide a main component for forming a light-emitting layer, or they may be a doping material in another main material, depending upon a combination of the above compounds with the organic compounds of the present invention.

The hole-injecting material is selected from compounds which are capable of transporting holes, are capable of receiving holes from the anode, have an excellent effect of injecting holes to a light-emitting layer or a light-emitting material, prevent the movement of excitons generated in a light-emitting layer to an electron-injecting zone or an electron-injecting material and have the excellent capability of forming a thin film. Suitable hole-injecting materials include for example a phthalocyanine derivative, a naphthalocyanine derivative, a porphyrin derivative, oxazole, oxadiazole, triazole, imidazole, imidazolone, imidazolthione, pyrazoline, pyrazolone, tetrahydroimidazole, oxazole, oxadiazole, hydrazone, acylhydrazone, polyarylalkane, stilbene, butadiene, benzidine type triphenylamine, styrylamine type triphenylamine, diamine type triphenylamine, derivatives of these, and polymer materials such as polyvinylcarbazole, polysilane and an electroconducting polymer.

In the organic EL device of the present invention, the hole-injecting material which is more effective is an aromatic tertiary amine derivative or a phthalocyanine derivative. Although not specially limited, specific examples of the tertiary amine derivative include triphenylamine, tritolylamine, tolyidiphenylamine, N,N′-diphenyl-N,N′-(3-methylphenyl)-1,1-biphenyl-4,4′-diamine, N,N,N′,N′-tetra(4-methylphenyl)-1,1′-phenyl-4,4′-diamine, N,N,N′,N′-tetra(4-methylphenyl)-1,1′-biphenyl-4,4′-diamine, N,N′-diphenyl-N,N′-di(1-naphthyl)-1,1′-biphenyl-4,4′-diamine, N,N′-di(methylphenyl)-N,N′-di(4-n-butylphenyl)-phenanthrene-9,10-diamine, 4,4′, 4″-tris(3-methylphenyl)-N-phenylamino)triphenylamine, 1,1-bis(4-di-p-tolylaminophenyl)cyclohexane, and oligomers or polymers having aromatic tertiary amine structures of these.

Although not specially limited, specific examples of the phthalocyanine (Pc) derivative include phthalocyanine derivatives or naphthalocyanine derivatives such as H₂Pc, CuPc, CoPc, NiPc, ZnPc, PdPc, FePc, MnPc, ClAlPC, ClGaPc, ClInPc, ClSnPc, Cl₂SiPc, (HO)AlPc, (HO)GaPc, VOPc, TiOPc, MoOPc, and GaPc-O—GaPc.

The hole transporting layer can reduce the driving voltage of the device and improve the confinement of the injected charge recombination within the light emitting layer, comprising the compounds of the present invention. Any conventional suitable aromatic amine hole transporting material described for the hole-injecting layer may be selected for forming this layer.

A preferred class of hole transporting materials is comprised of 4,4′-bis(9-carbazolyl)-1,1′-biphenyl compounds of the formula

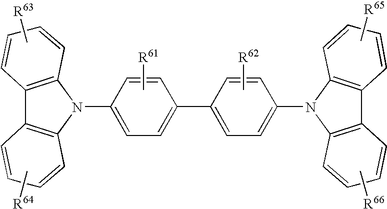

wherein R⁶¹ and R⁶² is a hydrogen atom or an C₁-C₃alkyl group; R⁶³ through R⁶⁶ are substituents independently selected from the group consisting of hydrogen, a C₁-C₆alkyl group, a C₁-C₆alkoxy group, a halogen atom, a dialkylamino group, a C₆-C₃₀aryl group, and the like. Illustrative examples of 4,4′-bis(9-carbazolyl)-1,1′-biphenyl compounds include 4,4′-bis(9-carbazolyl)-1,1′-biphenyl and 4,4′-bis(3-methyl-9-carbazolyl)-1,1′-biphenyl, and the like. The electron transporting layer is not necessarily required for the present device, but is optionally and preferably used for the primary purpose of improving the electron injection characteristics of the EL devices and the emission uniformity. Illustrative examples of electron transporting compounds, which can be utilized in this layer, include the metal chelates of 8-hydroxyquinoline as disclosed in U.S. Pat. Nos. 4,539,507, 5,151,629, and 5,150,006, the disclosures of which are totally incorporated herein by reference. Although not specially limited, specific examples of the metal complex compound include lithium 8-hydroxyquinolinate, zinc bis(8-hydroxyquinolinate), copper bis(8-hydroxyquinolinate), manganese bis(8-hydroxyquinolinate), aluminum tris(8-hydroxyquinolinate), aluminum tris(2-methyl-8-hydroxyquinolinate), gallium tris(8-hydroxyquinolinate), beryllium bis(10-hydroxybenzo[h]quinolinate), zinc bis(10-hydroxybenzo[h]quinolinate), chlorogallium bis(2-methyl-8-quinolinate), gallium bis(2-methyl-8-quinolinate)(o-cresolate), aluminum bis(2-methyl-8-quinolinate)(1-naphtholate), gallium bis(2-methyl-8-quinolinate)(2-naphtholate), gallium bis(2-methyl-8-quinolinate)phenolate, zinc bis(o-(2-benzooxazolyl)phenolate), zinc bis(o-(2-benzothiazolyl)phenolate) and zinc bis(o-(2-benzotrizolyl)phenolate). The nitrogen-containing five-membered derivative is preferably an oxazole, thiazole, thiadiazole, or triazole derivative. Although not specially limited, specific examples of the above nitrogen-containing five-membered derivative include 2,5-bis(1-phenyl)-1,3,4-oxazole, 1,4-bis(2-(4-methyl-5-phenyloxazolyl)benzene, 2,5-bis(1-phenyl)-1,3,4-thiazole, 2,5-bis(1-phenyl)-1,3,4-oxadiazole, 2-(4′-tert-butylphenyl)-5-(4″-biphenyl)1,3,4-oxadiazole, 2,5-bis(1-naphthyl)-1,3,4-oxadiazole, 1,4-bis[2-(5-phenyloxadiazolyl)]benzene, 1,4-bis[2-(5-phenyloxadiazolyl)-4-tert-butylbenzene], 2-(4′-tert-butylphenyl)-5-(4″-biphenyl)-1,3,4-thiadiazole, 2,5-bis(1-naphthyl)-1,3,4-thiadiazole, 1,4-bis[2-(5-phenylthiazolyl)]benzene, 2-(4′-tert-butylphenyl)-5-(4″-biphenyl)-1,3,4-triazole, 2,5-bis(1-naphthyl)-1,3,4-triazole and 1,4-bis[2-(5-phenyltriazolyl)]benzene. Another class of electron transport materials are oxadiazole metal chelates, such as bis[2-(2-hydroxyphenyl)-5-phenyl-1,3,4-oxadiazolato]zinc; bis[2-(2-hydroxyphenyl)-5-phenyl-1,3,4-oxadiazolato]beryllium; bis[2-(2-hydroxyphenyl)-5-(1-naphthyl)-1,3,4-oxadiazolato]zinc; bis[2-(2-hydroxyphenyl)-5-(1-naphthyl)-1,3,4-oxadiazolato]beryllium; bis[5-biphenyl-2-(2-hydroxyphenyl)-1,3,4-oxadiazolato]zinc; bis[5-biphenyl-2-(2-hydroxyphenyl)-1,3,4-oxadiazolato]beryllium; bis(2-hydroxyphenyl)-5-phenyl-1,3,4-oxadiazolato]lithium; bis[2-(2-hydroxyphenyl)-5-p-tolyl-1,3,4-oxadiazolato]zinc; b is 2-(2-hydroxyphenyl)-5-p-tolyl-1,3,4-oxadiazolato]beryllium; bis[5-(p-tert-butylphenyl)-2-(2-hydroxyphenyl)-1,3,4-oxadiazolato]zinc; bis[5-(p-tert-butylphenyl)-2-(2-hydroxyphenyl)-1,3,4-oxadiazolato]beryllium; bis[2-(2-hydroxyphenyl)-5-(3-fluorophenyl)-1,3,4-oxadiazolato]zinc; bis[2-(2-hydroxyphenyl)-5-(4-fluorophenyl)-1,3,4-oxadiazolato]zinc; bis[2-(2-hydroxyphenyl)-5-(4-fluorophenyl)-1,3,4-oxadiazolato]beryllium; bis[5-(4-chlorophenyl)-2-(2-hydroxyphenyl)-1,3,4-oxadiazolato]zinc; bis[2-(2-hydroxy phenyl)-5-(4-methoxyphenyl)-1,3,4-oxadiazolato]zinc; bis[2-(2-hydroxy-4-methylphenyl)-5-phenyl-1,3,4-oxadiazolato]zinc; bis[2-.alpha.-(2-hydroxynaphthyl)-5-phenyl-1,3,4-oxadiazolato]zinc; bis[2-(2-hydroxyphenyl)-5-p-pyridyl-1,3,4-oxadiazolato]zinc; bis[2-(2-hydroxyphenyl)-5-p-pyridyl-1,3,4-oxadiazolato]beryllium; bis[2-(2-hydroxyphenyl)-5-(2-thiophenyl)-1,3,4-oxadiazolato]zinc; bis[2-(2-hydroxyphenyl)-5-phenyl-1,3,4-thiadiazolato]zinc; bis[2-(2-hydroxyphenyl)-5-phenyl-1,3,4-thiadiazolato]beryllium; bis[2-(2-hydroxyphenyl)-5-(1-naphthyl)-1,3,4-thiadiazolato]zinc; and bis[2-(2-hydroxyphenyl)-5-(1-naphthyl)-1,3,4-thiadiazolato]beryllium, and the like.

In the organic EL device of the present invention, the light-emitting layer may contain, in addition to the light-emitting organic material of the present invention, at least one of other light-emitting material, other dopant, other hole-injecting material and other electron-injecting material. For improving the organic EL device of the present invention in the stability against temperature, humidity and ambient atmosphere, a protective layer may be formed on the surface of the device, or the device as a whole may be sealed with a silicone oil, or the like. The electrically conductive material used for the anode of the organic EL device is suitably selected from those materials having a work function of greater than 4 eV. The electrically conductive material includes carbon, aluminum, vanadium, iron, cobalt, nickel, tungsten, silver, gold, platinum, palladium, alloys of these, metal oxides such as tin oxide and indium oxide used for ITO substrates or NESA substrates, and organic electroconducting polymers, such as polythiophene and polypyrrole.

The electrically conductive material used for the cathode is suitably selected from those having a work function of smaller than 4 eV. The electrically conductive material includes magnesium, calcium, tin, lead, titanium, yttrium, lithium, ruthenium, manganese, aluminum and alloys of these, while the electrically conductive material shall not be limited to these. Examples of the alloys include magnesium/silver, magnesium/indium and lithium/aluminum, while the alloys shall not be limited to these. Each of the anode and the cathode may have a layer structure formed of two layers or more as required.

For the effective light emission of the organic EL device, at least one of the electrodes is desirably sufficiently transparent in the light emission wavelength region of the device. Further, the substrate is desirably transparent as well. The transparent electrode is produced from the above electrically conductive material by a deposition method or a sputtering method such that a predetermined light transmittance is secured. The electrode on the light emission surface side has for instance a light transmittance of at least 10%. The substrate is not specially limited so long as it has adequate mechanical and thermal strength and has transparency. For example, it is selected from glass substrates and substrates of transparent resins such as a polyethylene substrate, a polyethylene terephthalate substrate, a polyether sulfone substrate and a polypropylene substrate.

In the organic EL device of the present invention, each layer can be formed by any one of dry film forming methods such as a vacuum deposition method, a sputtering method, a plasma method and an ion plating method and wet film forming methods such as a spin coating method, a dipping method and a flow coating method. The thickness of each layer is not specially limited, while each layer is required to have a proper thickness. When the layer thickness is too large, inefficiently, a high voltage is required to achieve predetermined emission of light. When the layer thickness is too small, the layer is liable to have a pinhole, etc., so that sufficient light emission brightness is hard to obtain when an electric field is applied. The thickness of each layer is for example in the range of from about 5 nm to about 10 μm, for instance about 10 nm to about 0.2 μm.

In the wet film forming method, a material for forming an intended layer is dissolved or dispersed in a proper solvent such as ethanol, chloroform, tetrahydrofuran and dioxane, and a thin film is formed from the solution or dispersion. The solvent shall not be limited to the above solvents. For improving the film formability and preventing the occurrence of pinholes in any layer, the above solution or dispersion for forming the layer may contain a proper resin and a proper additive. The resin that can be used includes insulating resins such as polystyrene, polycarbonate, polyarylate, polyester, polyamide, polyurethane, polysulfone, polymethyl methacrylate, polymethyl acrylate and cellulose, copolymers of these, photoconductive resins such as poly-N-vinylcarbozole and polysilane, and electroconducting polymers such as polythiophene and polypyrrole. The above additive includes an antioxidant, an ultraviolet absorbent and a plasticizer.

When the light-emitting organic material of the present invention is used in a light-emitting layer of an organic EL device, an organic EL device can be improved in organic EL device characteristics such as light emission efficiency and maximum light emission brightness. Further, the organic EL device of the present invention is remarkably stable against heat and electric current and gives a usable light emission brightness at a low actuation voltage. The problematic deterioration of conventional devices can be remarkably decreased.

The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light.

The material of the present invention can be used in the fields of an organic EL device, an electrophotographic photoreceptor, a photoelectric converter, a solar cell, an image sensor, dye lasers and the like.

The following Examples illustrate the invention. In the Examples and throughout this application, the term light emitting material means the present triazine, or pyrimidine compounds.

### EXAMPLE 1

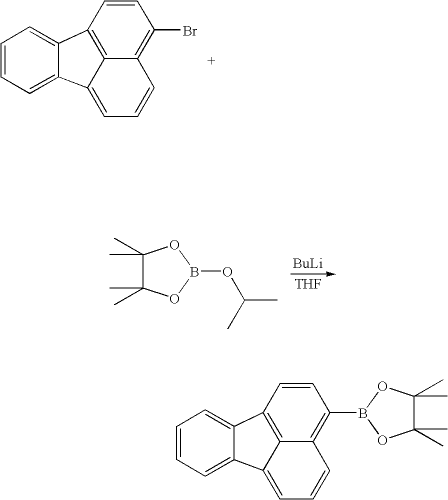

a) 3-bromo-fluoranthene is prepared as described in example 1 of DE 35 36 259. 2.00 g (7.11 mmol) 3-bromo-fluoranthene is dissolved in 40 ml anhydrous tetrahydrofuran (THF) under argon. To this solution 3.4 ml (8.54 mmol) n-butyl lithium are added at −78° C. After 1 h 2.65 g (14.2 mmol) 2-isopropoxy-4,4,5,5-tetramethyl-1,3,2-dioxaborolane are added at −78° C. The reaction mixture is stirred at −78° C. for 1 h. The reaction mixture is warmed up to 20° C., poured into water and extracted with 95% dichloromethane and 5% ether. The organic phase is dried with magnesium sulphate and the solvent is removed in vacuum. The product is used without further purification for the next reaction.

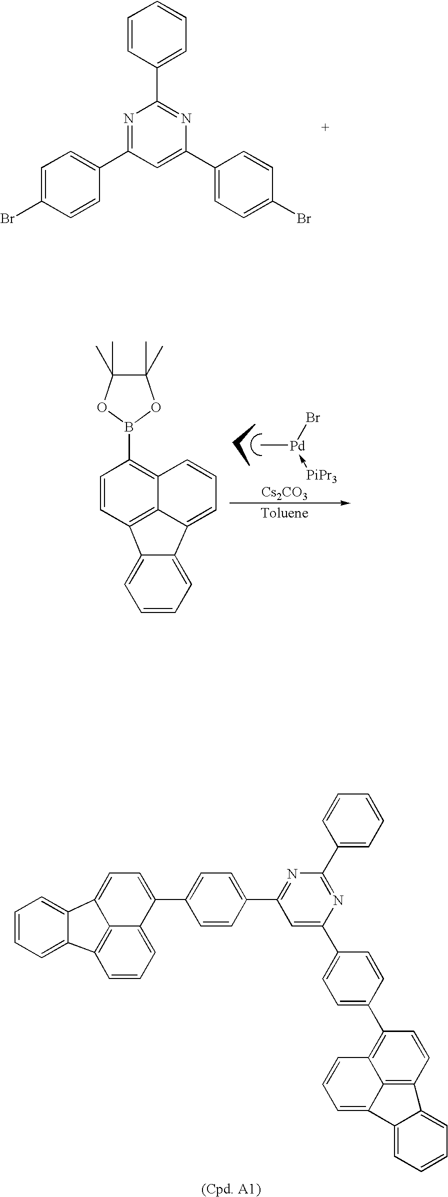

b) To 1.00 g (2.15 mmol) of 4,6-bis-(4-bromo-phenyl)-2-phenyl-pyrimidine in 50 ml toluene and 10 ml water, 3.29 g (10.1 mmol) Cs₂CO₃ are added under an argon atmosphere. The palladium catalyst is added. Then 2.11 g (6.44 mmol) 4,4,5,5-tetramethyl-1,3,2-dioxaborolan-3-fluoranthene is added and the reaction mixture is refluxed for 21 h under argon. The reaction mixture is filtered on HYFLO® Super Gel (Fluka). The organic phase is washed with water and dried with magnesium sulphate and the HYFLO® is washed with dichloromethane. The solvent is removed in vacuum. A column chromatography on silica gel with toluene/hexane 1/1 gives the desired product.

Melting point 310-312° C.

### EXAMPLE 2

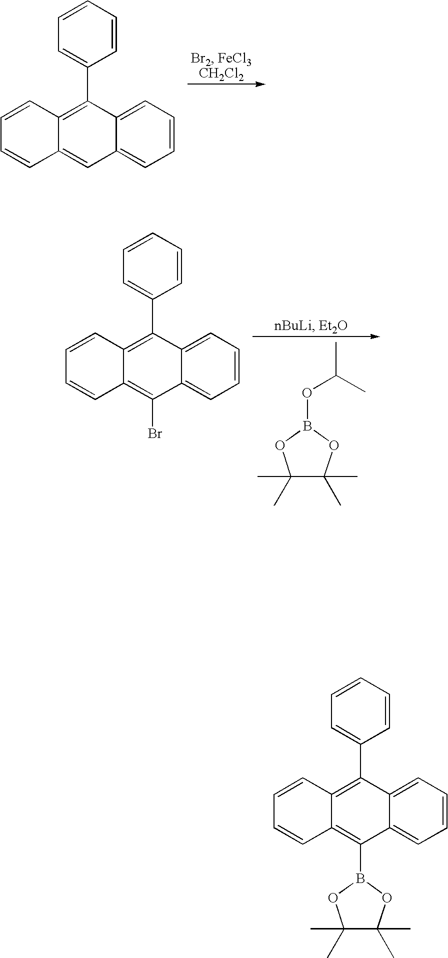

a) 9-(10-phenylanthryl)-4,4,5,5-tertramethyl-1,3,2-dioxaborolane is prepared according to example 5 of US-A-20020132134.

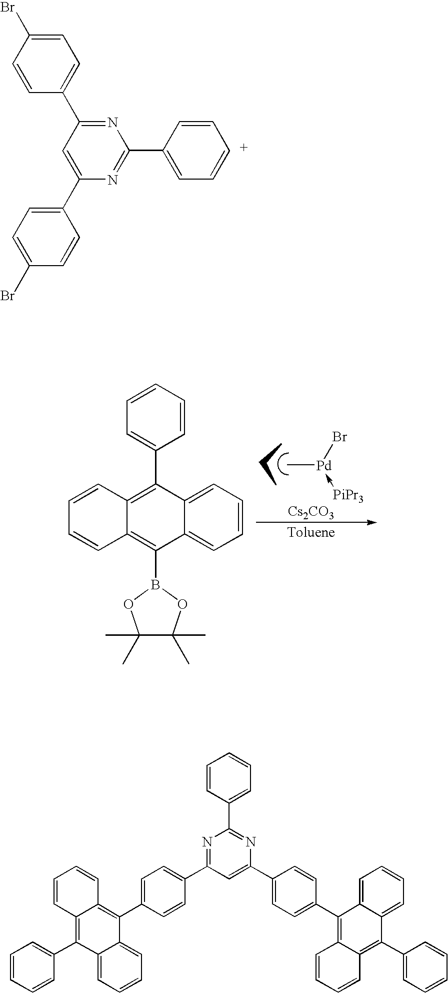

The desired pyrimidine compound is prepared in analogy to example 1b).

### APPLICATION EXAMPLE 1

Present compound A1, as light emitting material, 2,5-bis(1-naphthyl)-1,3,4-oxadiazole and a polycarbonate resin in a weight ratio of 5:3:2 are dissolved in tetrahydrofuran, and the solution is spin-coated on a cleaned glass substrate with an ITO electrode to form a light-emitting layer having a thickness of 100 nm. An electrode having a thickness of 150 nm is formed thereon from a magnesium/indium alloy having a magnesium/indium mixing ratio of 10/1, to obtain an organic EL device. The device exhibits light emission with excellent brightness and efficiency at a direct current voltage of 5 V.

### APPLICATION EXAMPLE 2

Present compound A1 is vacuum-deposited on a cleaned glass substrate with an ITO electrode to form a light-emitting layer having a thickness of 100 nm. An electrode having a thickness of 100 nm is formed thereon from a magnesium/silver alloy having a magnesium/silver mixing ratio of 10/1, to obtain an organic EL device. The light-emitting layer is formed by deposition under a vacuum of 10⁻⁶ Torr at a substrate temperature of room temperature. The device shows emission having an excellent brightness and efficiency at a direct current voltage of 5 V.

### APPLICATION EXAMPLE 3

Present compound A1 is dissolved in methylene chloride tetrahydrofuran, and the solution is spin-coated on a cleaned glass substrate with an ITO electrode to form a light-emitting layer having a thickness of 50 nm. Then, aluminum bis(2-methyl-8-quinolinate)(2-naphtolate) is vacuum-deposited to form an electron transporting layer having a thickness of 10 nm, and an electrode having a thickness of 100 nm is formed thereon from a magnesium/aluminum alloy having a magnesium/aluminum mixing ratio of 10/1, to obtain an organic EL device. The light-emitting layer and the electron-injecting layer are formed by deposition under a vacuum of 10⁻⁶ Torr at a substrate temperature of room temperature. The device shows an emission having an excellent brightness and efficiency at a direct current voltage of 5 V.

### APPLICATION EXAMPLE 4

One of hole transporting materials (H-1) to (H-6) is vacuum-deposited on a cleaned glass substrate with an ITO electrode, to form a hole transporting layer having a thickness of 30 nm. Then, present compound A1 is vacuum-deposited to form a light-emitting layer having a thickness of 30 nm. Further, one of electron transporting materials (E-1) to (E-6) is vacuum-deposited to form an electron transporting layer having a thickness of 30 nm. An electrode having a thickness of 150 nm is formed thereon from a magnesium/silver alloy having a magnesium/silver mixing ratio of 10/1, to obtain an organic EL device. Each layer is formed under a vacuum of 10⁶ Torr at a substrate temperature of room temperature. All the organic EL devices obtained in these Examples shows high brightness and efficiency.

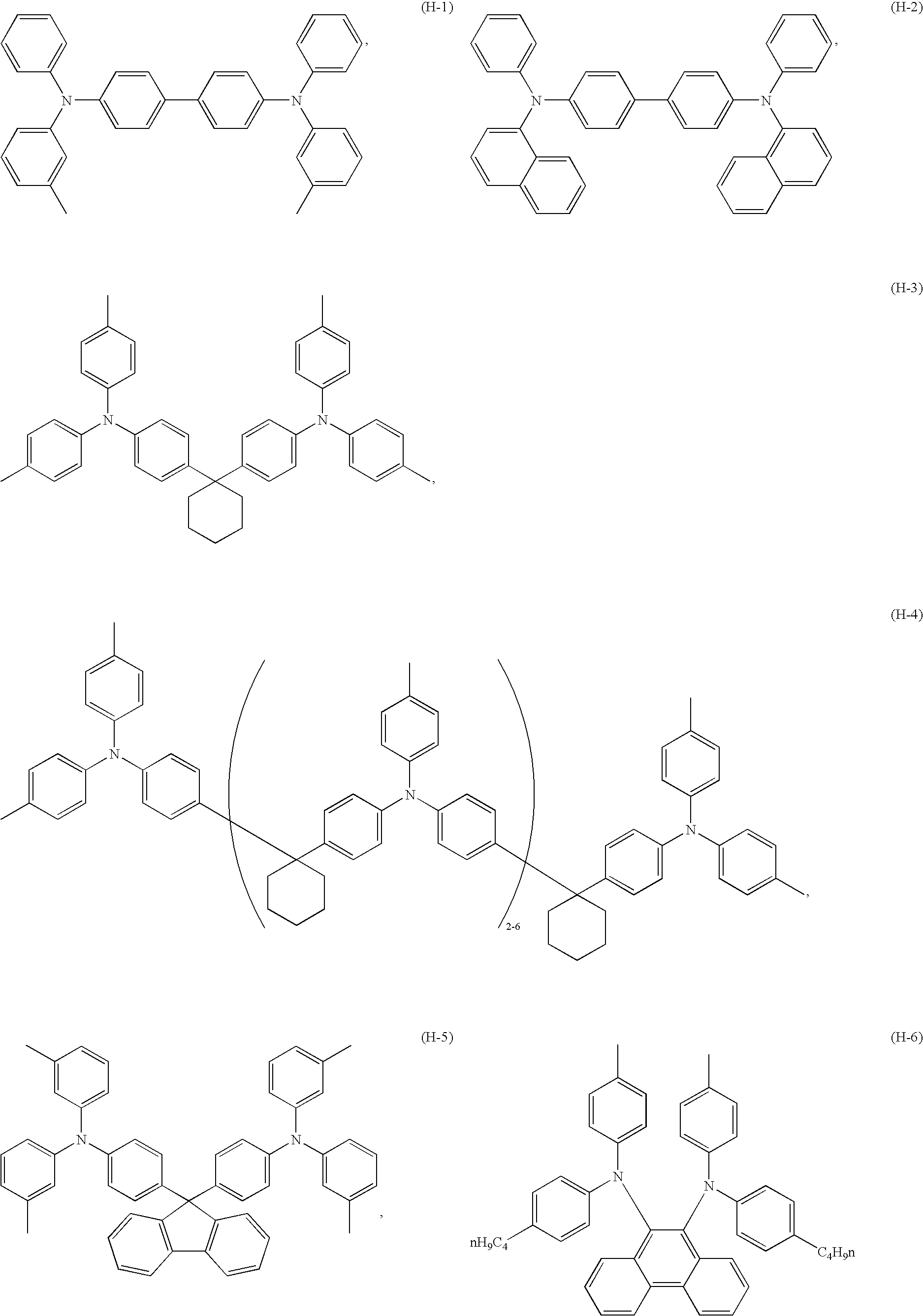

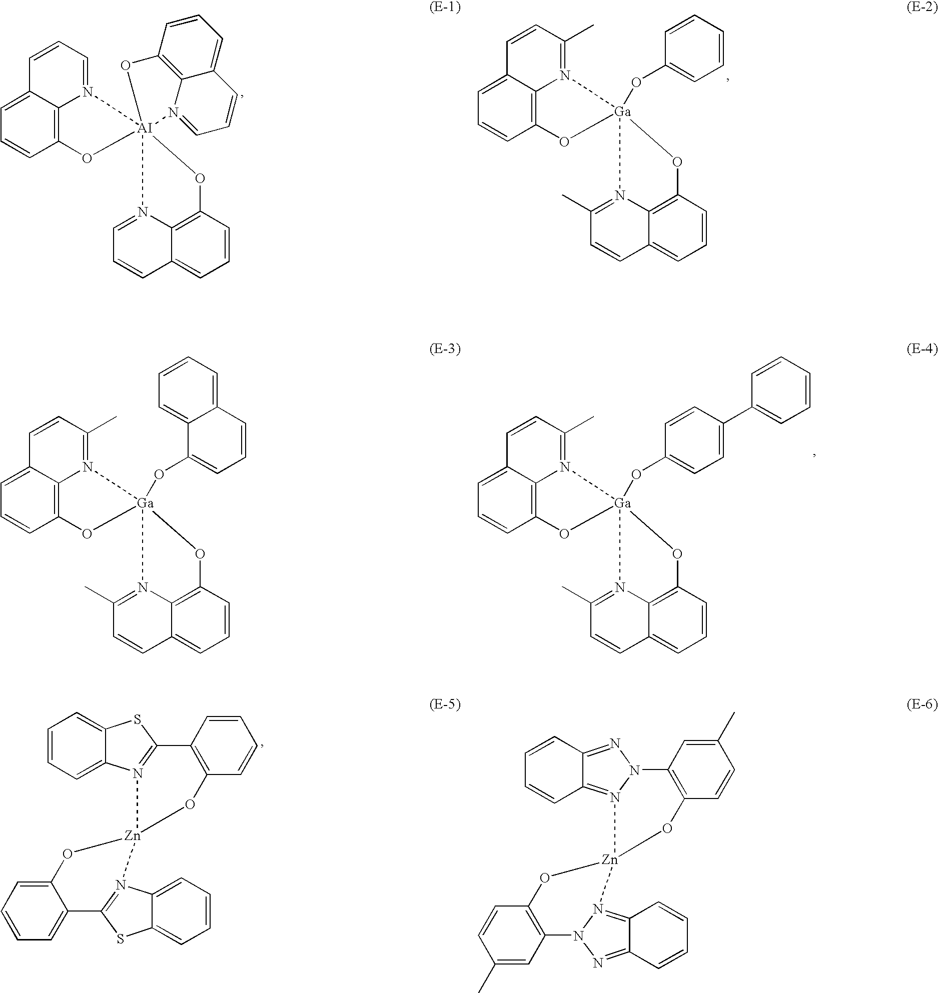

### APPLICATION EXAMPLE 5

On a cleaned glass substrate with an ITO electrode, 4,4′,4″-tris(N-(3-methylphenyl)-N-phenylamino)triphenylamine is vacuum-deposited to form a hole-injecting layer having a thickness of 25 nm. Further, a hole transporting material (H-1) is vacuum-deposited to form a hole transporting layer having a thickness of 5 nm. Then, compound A1 as light-emitting material is vacuum-deposited to form a light-emitting layer having a thickness of 20 nm. Further, an electron transporting material (E-1) is vacuum-deposited to form an electron transporting layer having a thickness of 30 nm. Then, an electrode having a thickness of 150 nm is formed thereon from a magnesium/silver alloy having an magnesium/silver mixing ratio of 10/1, to obtain an organic EL device. The device shows emission having an outstanding brightness and efficiency at a direct current voltage of 5 V.

### APPLICATION EXAMPLE 6

A hole transporting material (H-5) is vacuum-deposited on a cleaned glass substrate with an ITO electrode to form a hole transporting layer having a thickness of 20 nm. Then, compound A1 as light-emitting material is vacuum-deposited to form a light-emitting layer having a thickness of 20 nm. Further, an electron transporting material (E-2) is vacuum-deposited to form a first electron transporting layer having a thickness of 20 nm. Then, an electron transporting material (E-5) is vacuum-deposited to form a second electron transporting layer having a thickness of 10 nm, and an electrode having a thickness of 150 nm is formed thereon from a magnesium/silver alloy having an magnesium/silver mixing ratio of 10/1, to obtain an organic EL device. The device shows light emission having an excellent brightness and efficiency at a direct current voltage of 5 V.

### APPLICATION EXAMPLE 7

An organic EL device is prepared in the same manner as in Application Example 4 except that the light-emitting layer is replaced with a 30 nm thick light-emitting layer formed by vacuum-depositing compound A1 and one of the dopant compounds (D-1) to (D-7) in a weight ratio of 100:1. All the organic EL devices obtained in these Examples shows high brightness characteristics and gives intended light emission colors.

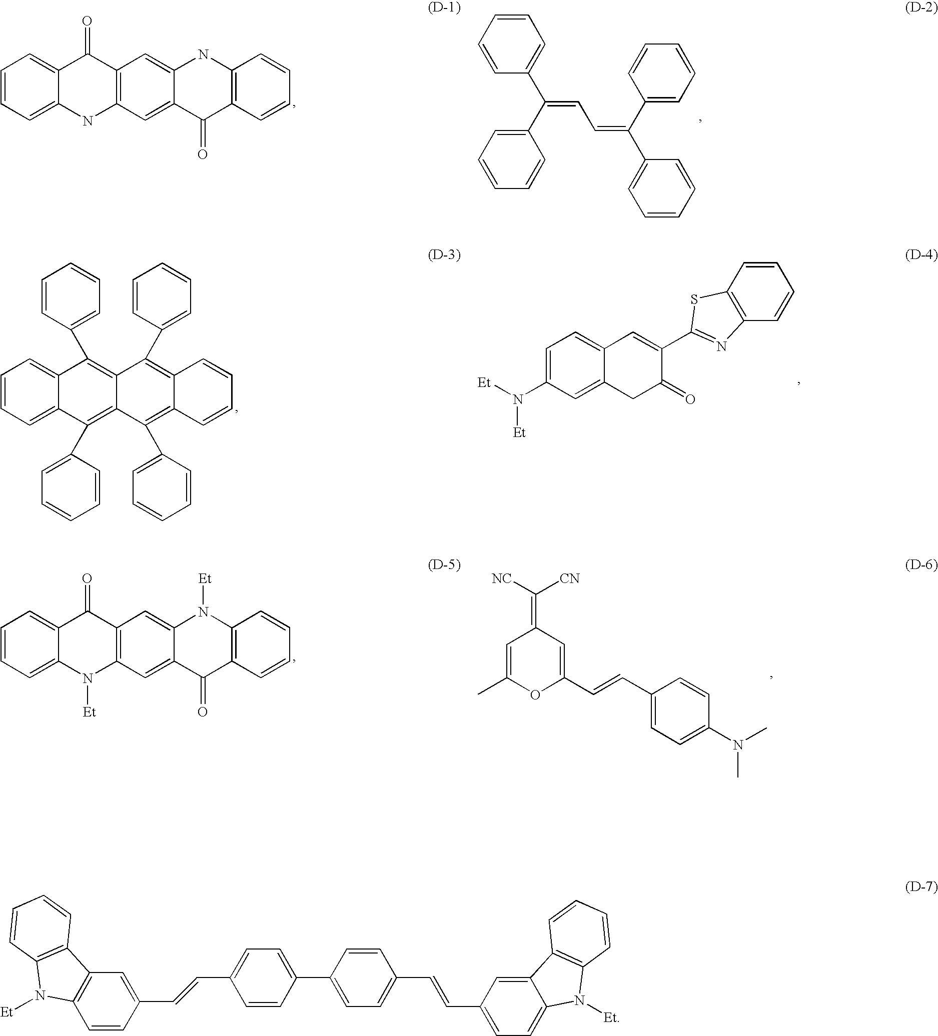

### APPLICATION EXAMPLE 8

On a cleaned glass substrate with an ITO electrode, N,N′-1-naphthyl-N,N′-diphenyl-1,1′-biphenyl-4,4′-diamine and 5,10-diphenylanthracene are vacuum-deposited to form a hole-injecting layer. Further, 4,4′-bis(9-carbazolyl)-1,1′-biphenyl is vacuum-deposited to form a hole transporting layer. Then, compound A1 as light-emitting material is vacuum-deposited to form a light-emitting layer. Then, an electrode is formed thereon from a magnesium/silver alloy having an magnesium/silver mixing ratio of 9/1, to obtain an organic EL device. The device shows emission having an outstanding brightness and efficiency at a direct current voltage of 5 V.

The organic EL devices obtained in the Application Examples of the present invention show an excellent light emission brightness and achieved a high light emission efficiency. When the organic EL devices obtained in the above Examples are allowed to continuously emit light at 3 (mA/cm²), all the organic EL devices remain stable. Since the light-emitting materials of the present invention have a very high fluorescence quantum efficiency, the organic EL devices using the light-emitting materials achieved light emission with a high brightness in a low electric current applied region, and when the light-emitting layer additionally uses a doping material, the organic EL devices are improved in maximum light emission brightness and maximum light emission efficiency. Further, by adding a doping material having a different fluorescent color to the light-emitting material of the present invention, there are obtained light-emitting devices having a different light emission color. The organic EL devices of the present invention accomplish improvements in light emission efficiency and light emission brightness and a longer device life, and does not impose any limitations on a light-emitting material, a dopant, a hole transporting material, an electron transporting material, a sensitizer, a resin and an electrode material used in combination and the method of producing the device. The organic EL device using the material of the present invention as a light-emitting material can achieve light emission having a high brightness with a high light emission efficiency and a longer life as compared with conventional devices. According to the light-emitting material of the present invention and the organic EL device of the present invention, there can be achieved an organic EL device having a high brightness, a high light emission efficiency and a long life.

1. A compound of formula 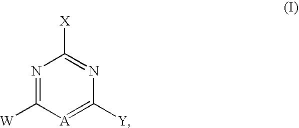
wherein
A is CH, or N,
X is a group of the formula —(X¹)ₘ—(X²)ₙ—X³
W is a group of the formula —(W¹)ₐ—(W²)_{b}—W³, and
Y is a group of the formula —(Y¹)_{c}—(Y²)_{d}—Y³, wherein
a, b, c, d, m and n are independently of each other 0, or 1,
W¹, W², X¹, X², Y¹ and Y² are independently of each other a group of formula 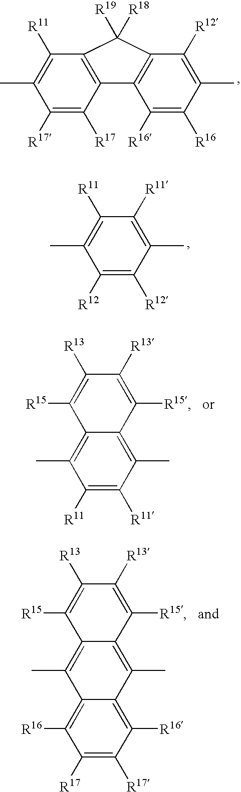
W³, X³ and Y³ are independently of each other a group of formula 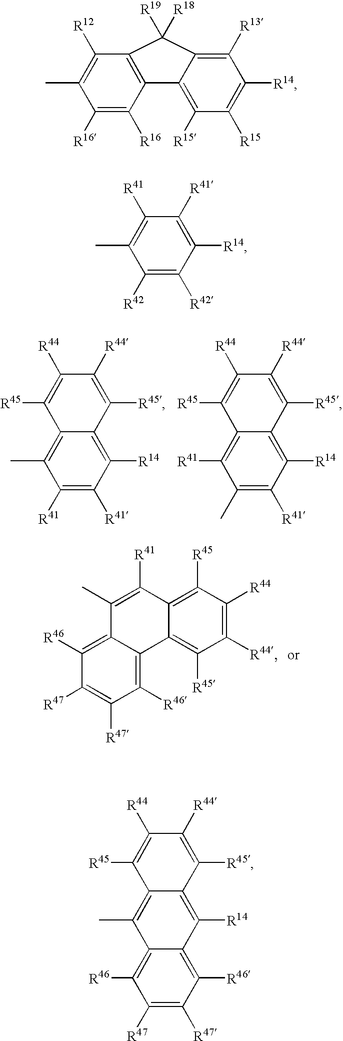
or a C₁₆-C₃₀aryl group, which can be substituted by G;
R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by G; C₁-C₁₈alkyl; C₁-C₁₈alkyl which is substituted by E and/or interrupted by D; C₁-C₁₈alkoxy; or C₁-C₁₈alkoxy which is substituted by E and/or interrupted by D; C₇-C₁₈aralkyl; or C₇-C₁₈aralkyl which is substituted by G;
R¹⁴ is H, C₁-C₁₈alkyl; or C₁-C₁₈alkyl which is substituted by E and/or interrupted by D; C₁-C₁₈alkoxy; or
C₁-C₁₈alkoxy which is substituted by E and/or interrupted by D; 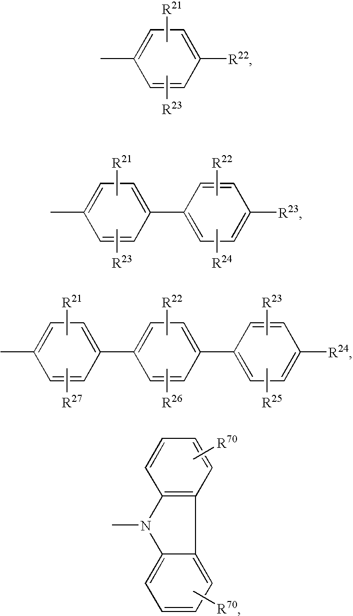
or —NAr¹Ar², wherein Ar¹ and Ar² are independently of each other 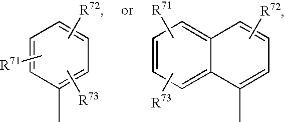
R²¹, R²², R²³, R²⁴, R²⁵, R²⁶ and R²⁷ are independently of each other H, E, C₁-C₁₈alkyl; C₁-C₁₈alkyl which is substituted by E and/or interrupted by D; C₇-C₁₈aralkyl; C₇-C₁₈aralkyl which is substituted by G;
R⁷⁰, R⁷¹, R⁷² and R⁷³ are independently of each other C₁-C₁₈alkyl, C₁-C₁₈alkyl which is interrupted by —O—, C₆-C₁₈aryl; C₆-C₁₈aryl which is substituted by C₁-C₁₈alkyl, —OR⁵, or —SR⁵,
R¹⁸ and R¹⁹ are independently of each other C₁-C₁₈alkyl; C₁-C₁₈alkoxy, C₆-C₁₈aryl; C₇-C₁₈aralkyl; or R¹⁸ and R¹⁹ together form a five- or six-membered ring, which can optionally be substituted by C₁-C₈alkyl,
D is —CO—; —COO—; —OCOO—; —S—; —SO—; —SO₂—; —O—; —NR⁵—; SiR⁶¹R⁶²—; —POR⁵—; —CR⁶³═CR⁶⁴—; or —C≡C—;
E is —OR⁵; —SR⁵; —NR⁵R⁶; —COR⁸; —COOR⁷; —OCOOR⁷, —CONR⁵R⁶; —CN; or halogen;
G is E, or C₁-C₁₈alkyl,
wherein R⁵ and R⁶ are independently of each other C₆-C₁₈aryl; C₆-C₁₈aryl which is substituted by C₁-C₁₈alkyl, or C₁-C₁₈alkoxy; C₁-C₁₈alkyl or C₁-C₁₈alkyl which is interrupted by —O—; or
R⁵ and R⁶ together form a five or six membered ring,
R⁷ is C₇-C₁₂alkylaryl; C₁-C₁₈alkyl; or C₁-C₁₈alkyl which is interrupted by —O—;
R⁸ is C₆-C₁₈aryl; C₆-C₁₈aryl which is substituted by C₁-C₁₈alkyl, or C₁-C₁₈alkoxy; C₁-C₁₈alkyl, C₇-C₁₂alkylaryl, or C₁-C₁₈alkyl which is interrupted by —O—;
R⁶¹ and R⁶² are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, C₁-C₁₈alkoxy; or C₁-C₁₈alkyl which is interrupted by —O—.

2. The compound according to claim 1, wherein R¹¹, R^{11′}, R¹², R^{12′}, R¹³, R^{13′}, R¹⁵, R^{15′}, R¹⁶, R^{16′}, R¹⁷ and R^{17′}, R⁴¹, R^{41′}, R⁴², R^{42′}, R⁴⁴, R^{44′}, R⁴⁵, R^{45′}, R⁴⁶, R^{46′}, R⁴⁷, and R^{47′} as well as R¹⁴ are independently of each other H, E; C₁-C₁₈alkyl; C₁-C₁₈alkyl which is substituted by E and/or interrupted by D, C₆-C₂₄aryl, C₆-C₂₄aryl which is substituted by G; wherein D is —O—, E is —OR⁵;
—SR⁵; —NR⁵R⁶; —COR⁸; —COOR⁷; —CONR⁵R⁶; —CN; —OCOOR⁷; or halogen; G is E, or C₁-C₈alkyl; wherein
R⁵ and R⁶ are independently of each other C₆-C₁₂aryl, or C₁-C₈alkyl;
R⁷ is C₇-C₁₂alkylaryl, or C₁-C₈alkyl; and
R⁸ is C₆-C₁₂aryl; or C₁-C₈alkyl.

3. The compound according to claim 1 of formula 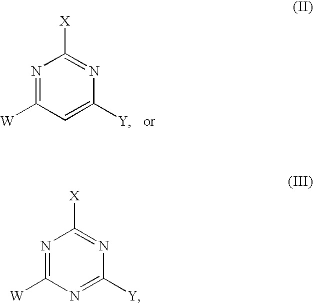
wherein
X is 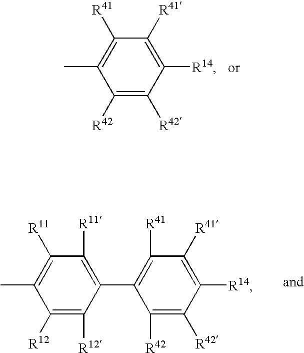
W and Y are a group of the formula —W¹—W²—W³, wherein
W¹ is a group of formula 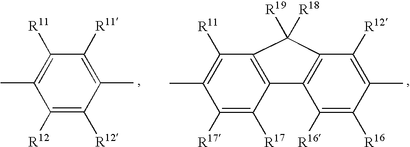
W² is a group of formula 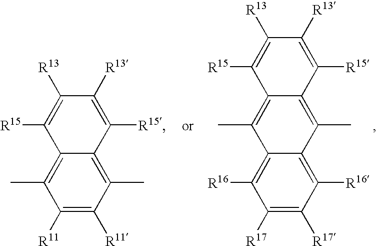
W³ is a group of formula 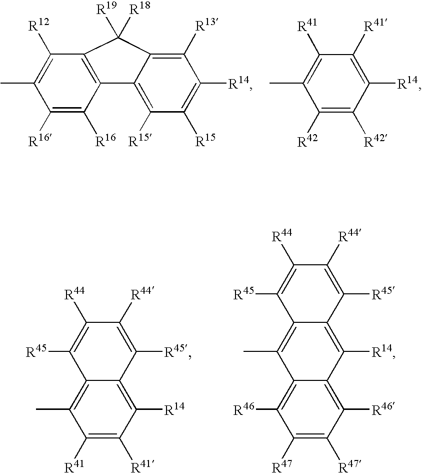
wherein
R¹¹, R^{11′}, R¹², R^{12′}, R¹³. R^{13′}, R¹⁴, R¹⁵, R^{15′}, R¹⁶, R^{16′}, R¹⁷, R^{17′}, R¹⁸, R¹⁹, R⁴¹, R^{41′}, R⁴², R^{42′}, R⁴⁴, R^{44′}, R⁴⁵, R^{45′}, R⁴⁶, R^{46′}, R⁴⁷ and R^{47′} are as defined in claim 1, or X, W and Y are a group of the formula —W¹—W²—W³, wherein W¹, W² and W³ are as defined above.

4. The compound according to claim 3 of formula II, or III, wherein
X is 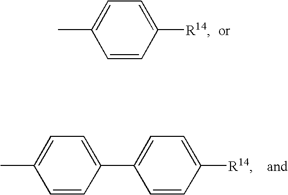
W and Y are a group of the formula —W¹—W²—W³, wherein
W¹ is a group of formula 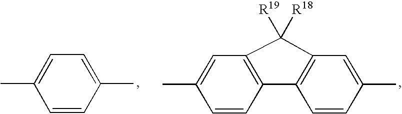
W² is a group of formula 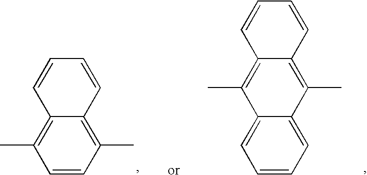
W³ is a group of formula 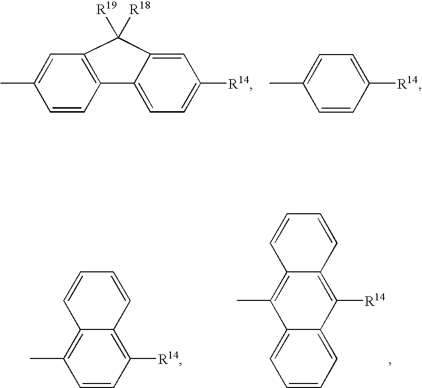
wherein
R¹⁴ is H, C₁-C₈alkyl, or C₁-C₈alkoxy, and
wherein R¹⁸ and R¹⁹ are independently of each other C₁-C₈alkyl, or cyclohexyl.

5. The compound according to claim 1 of formula 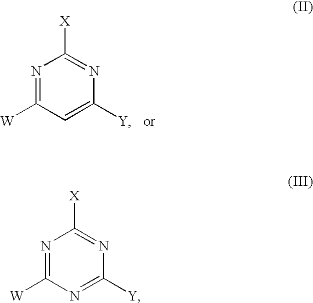
wherein
X is 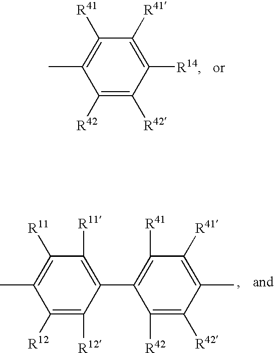
W and Y are a group Ar¹—Ar², wherein
Ar¹ is a group of formula 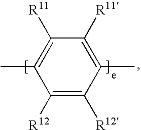
Ar² is a group of formula 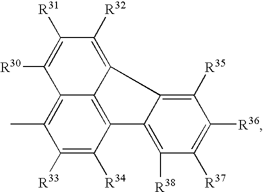
wherein
R³⁰, R³¹, R³², R³³, R³⁴, R³⁵, R³⁶, R³⁷ and R³⁸ are independently of each other H, E, C₆-C₁₈aryl; C₆-C₁₈aryl which is substituted by G; C₁-C₁₈alkyl; C₁-C₁₈alkyl which is substituted by E and/or interrupted by D; C₇-C₁₈aralkyl; or C₇-C₁₈aralkyl which is substituted by G;
e is an integer 1, or 2, or
X, W and Y are independently of each other a group Ar¹—Ar², wherein Ar¹ and Ar² are as defined above.

6. The compound of formula II or III according to claim 5, wherein
X is 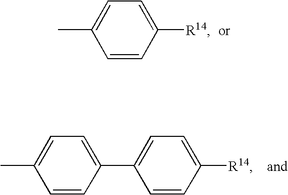
W and Y are a group Ar¹—Ar², wherein
Ar¹ is a group of formula 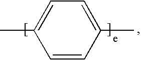
Ar² is a group of formula 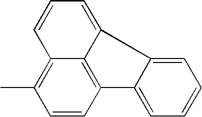
e is an integer 1, or 2,
R¹⁴ is H, C₁-C₈alkyl, or C₁-C₈alkoxy, or
X, W and Y are independently of each other a group Ar¹—Ar², wherein Ar¹ and Ar² are as defined above.

7. The compound according to claim 1 of formula 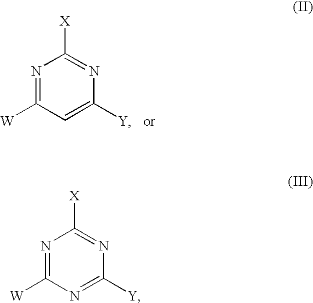
wherein
X is 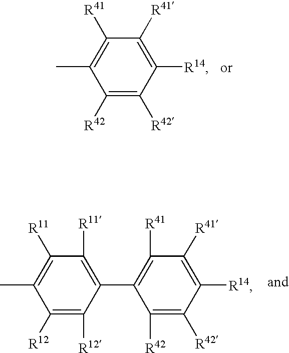
W and Y are a group of the formula —W¹—(W²)_{b}—W³, wherein b is 1, or 2,
W¹ and W² are independently of each other a group of formula 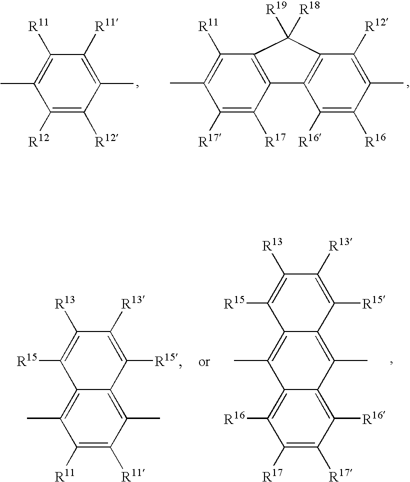
W³ is a group of formula 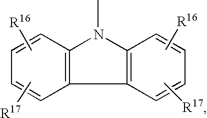
or —NR⁵⁰R⁵¹, wherein R⁵⁰ and R⁵¹ are independently of each other a group of formula 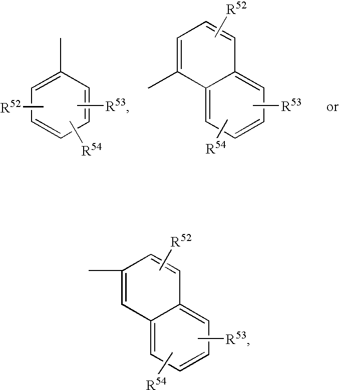
wherein R⁵², R⁵³ and R⁵⁴ are independently of each other hydrogen, C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group or a siloxanyl group, or X, W and Y are independently of each other a group of the formula —W¹—(W²)_{b}—W³, wherein b, W¹, W² and W³ are as defined above.

8. The compound according to claim 7 of formula II, or III, wherein
X is 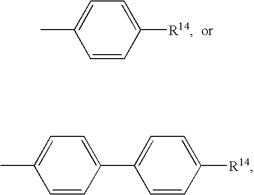
and
W and Y are a group of the formula —W¹—(W²)_{b}—W³, wherein b is 1, or 2,
W¹ is a group of formula 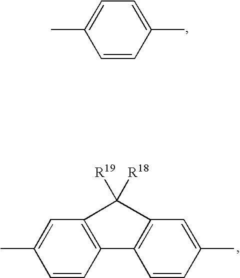
W² is a group of formula 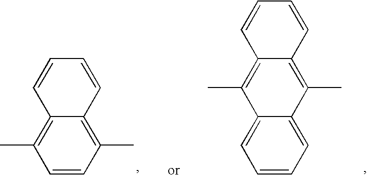
W³ is a group of formula 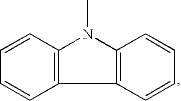
or —NR⁵⁰R⁵¹, wherein R⁵⁰ and R⁵¹ are independently of each other a group of formula 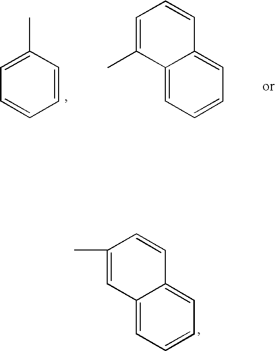
R¹⁴ is H, C₁-C₈alkyl, or C₁-C₈alkoxy, and
R¹⁸ and R¹⁹ are independently of each other C₁-C₈alkyl.

9. An electroluminescent device, comprising a compound of formula I according to claim 1.

10. Electroluminescent device according to claim 9, wherein the electroluminescent device comprises in this order
(a) an anode
(b) a hole injecting layer and/or a hole transporting layer
(c) a light-emitting layer
(d) optionally an electron transporting layer and
(e) a cathode.

11. Electroluminescent device according to claim 10, wherein the compound of formula I forms the light-emitting layer.

12. An electrophotographic photoreceptor, photoelectric converter, solar cell, image sensor, dye laser or electroluminescent device containing a compound of formula I according to claim 1.

13. The compound according to claim 1, wherein the C₁₆-C₃₀aryl group is a fluoranthenyl, triphenlenyl, chrysenyl, naphthacenyl, picenyl, perylenyl, pentaphenyl, hexacenyl, or pyrenyl group which can be substituted by G.

14. The compound according to claim 1, wherein when R⁵ and R⁶ together form a five or six membered ring is

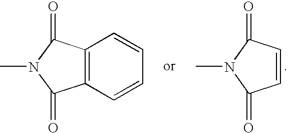

15. The compound according to claim 2, wherein R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷, and R^{47′} as well as R¹⁴ are independently of each other H or E.
